(19) United States
(12) Patent Application Publication
(10) Pub. No.: US 2010/0266558 A1
(43) Pub. Date: Oct. 21, 2010

(54) METHOD AND ASSAY FOR GLYCOSYLATION PATTERN DETECTION RELATED TO CELL STATE OF STEM CELLS

(76) Inventor: Dov Zipori, Rehovot (IL)

Correspondence Address: DR. D. GRAESER LTD., 9003 FLORIN WAY, UPPER MARLBORO, MD, 20772, US

(21) Appl. No.: 12/808,678

(22) PCT Filed: Dec. 17, 2008
(86) PCT No.: PCT/IL08/01628

§ 371 Date: Jun. 17, 2010

Related U.S. Application Data
(60) Provisional application No. 61/006,080, filed on Dec. 18, 2007.

(51) Int. Cl. A61K 35/12; G01N 33/53; C40B 30/04

(52) U.S. Cl. 424/93.7; 435/7.2; 506/9

(57) ABSTRACT

A method and assay for characterizing populations of stem cells according to their glycosylation pattern, particularly for distinguishing between stem cell populations, for example with regard to state of differentiation.

### FIELD OF THE INVENTION

The present invention relates to a method and assay for detecting glycosylation patterns of stem cells, and in particular, to such a method and assay which enable the state of a mesenchymal stem cell, particularly with regard to differentiation, to be determined according to the detected glycosylation pattern.

### BACKGROUND OF THE INVENTION

Oligosaccharides and polysaccharides are polymers that consist of monosaccharide (sugar) units, connected to each other via glycosidic bonds. These polymers have a structure that can be described in terms of the linear sequence of the monosaccharide subunits, which is known as the two-dimensional structure of the polysaccharide. Polysaccharides can also be described in terms of the structures formed in three dimensions by their component monosaccharide subunits.

The saccharide chain has, like a chain of DNA or protein, two dissimilar ends. In the case of saccharide chains, these are the reducing end (corresponding to the aldehyde group of the linear sugar molecule) and the non-reducing end. Unlike proteins and DNA, however, polysaccharides are generally branched, with essentially each of the sugar units in the polysaccharide serving as an optional branching point, resulting in complex structures with diversity at both the level of the monomers and of the linkage.

Glycosylation, the addition of covalently bound monosaccharides or extended sugar chains to proteins, is one of four chief co-translational and post-translational modifications; which, take place during the synthesis of membrane and secreted glycoproteins. Glycosylation proceeds via a stepwise addition or removal of individual glycosides, forming linear or branched chains. The structure of the glycans is dependent on the structure of the protein, onto which it is built (Haltiwanger and Lowe, 2004). The two principle types of protein glycosylation are N-glycosylation and O-glycosylation. Glycoproteins reside in the extracellular matrix and fluids; as well as inside cells, both in the cytoplasm, cellular organelles and cell membranes. A glycosylation may result in significant modifications in protein conformation, which might lead to alterations in protein functions and interactions. Glycosylation sites, in each glycoprotein, may vary both in the proportion of glycans and in their structures. Thus, each glycoprotein is actually a heterogeneous mixture of so-called glycoforms. The substantial structural variety of glycans found in glycoproteins, is accounted by additional factors. First, oligosaccharides are synthesized in a non-template process without proofreading. Second, the process entails a synchronized activity of many enzymes, including: glycosidases, glycan phosphorylases, polysaccharide lyases and glycosyltransferases. Third, the availability of these enzymes might vary throughout cell growth, differentiation and development (Geyer and Geyer, 2006).

There are a number of proteins that bind to saccharides. Many of these proteins bind specifically to a certain short mono or disaccharide sequence.

Lectins are a broad family of proteins that bind saccharides. A large number of plant lectins have been characterized and are used in research. Many mammalian lectins have also been characterized.

Antibodies are proteins that specifically recognize certain molecular structures. Antibodies may also recognize saccharide structures, as do lectins.

Glycosidases are enzymes that cleave glycosidic bonds within the saccharide chain. Also glycosidases may recognize certain oligosaccharide sequences specifically.

Glycosyltransferases are nucleotide sugar-dependent enzymes, which use sugar donors containing a nucleoside phosphate or a lipid phosphate leaving group; by which they catalyze glycosidic bond formation. In vivo, these acceptor molecules are the growing glycan structures. Thus far, 3-D structures of glycosyltransferases have revealed only two structural folds, GT-A and GT-B (Lairson et al, 2008).

The structural determination of polysaccharides is of fundamental importance for the development of glycobiology. Research in glycobiology relates to subjects as diverse as the bacterial cell walls, blood glycans, to growth factor and cell surface receptor structures involved in viral disease, such as HIV infection, autoimmune diseases such as insulin-dependent diabetes and rheumatoid arthritis, and abnormal cell growth as it occurs in cancer.

The importance of glycomolecules is highlighted by the discovery of penicillin, an inhibitor of glycomolecule synthesis in the bacterial cell-wall and possibly the most successful antibiotic discovered to date.

Another example is the medical use of heparin, a glycosaminoglycan that inhibits blood clotting and is today widely used in medicine. Further examples of medically-important glycomolecules include: glycosaminoglycans (GAGs), heparan sulphate, monoclonal antibodies, cytokines (e.g. IL-8, TNF, and the blockbuster EPO), chemokines (e.g. acidic fibroblast growth factor) and various growth factors. The aforementioned cytokines, chemokines and growth factors are also capable of binding to GAGs and other polysaccharides, and therefore may also be considered to be lectins.

The structural complexity of polysaccharides has hindered their analysis. In the absence of structural information, the researcher must assume that the building units are selected from any of the saccharide units known today. In addition, these units may have been modified, during synthesis, e.g., by the addition of sulfate groups. Without the ability to measure such carbohydrate structural information, the researcher cannot determine the true, correct glycosylation pattern for populations of cells, for example in a tissue. In addition, these units may have been modified, e.g. by the addition of sulfate groups, during synthesis, such that merely understanding which types of saccharides may have been added does not provide a complete picture.

Furthermore, the connections between saccharide units are multifold. A saccharide may be connected to any of the C1, C2, C3, C4, or C6 atoms if the sugar unit to which it is connected is a hexose. Moreover, the connection to the C1 atom may be in either alpha or beta configuration. In addition, the difference in structure between many sugars is minute, as a sugar unit may differ from another merely by the position of the hydroxyl groups (epimers).

Protein glycosylation analysis is generally performed by analyzing the glycans following their release from the glycoprotein. Combinations of chromatographic and mass-spectrometric techniques are usually employed for analysis. This process is labor-intensive, and preparation of samples may take days to weeks. The analyses require large amounts of purified material, sophisticated equipment and a high level of expertise. Therefore glycoanalysis is not readily available to all biological researchers. In addition to these difficulties, application of all of the above methods to complex glycoprotein mixtures, such as sub-cellular fractions, is difficult even for the glycoanalysis experts, and only a limited success has been reported in the literature.

In vivo, glycosylation is tissue dependant and can vary significantly with cell state. In vitro, glycosylation strongly depends on growth conditions: the type of cell, nutrient concentrations, pH, cell density, and age can affect the glycosylation patterns of glycoproteins. The number of glycoforms and their relative abundance within a cell are affected by the intrinsic structural properties of the individual protein, as well as the repertoire of glycosylation enzymes available (including their type, concentration, kinetic characteristics, compartmentalization). This repertoire has been shown to change upon changes in cell state (e.g. oncogenic transformation).

Cell surface glycoproteins are important in cell communication and differentiation (Crocker and Feizi, 1996). Glycan expression is cell type specific (Haltiwanger and Lowe, 2004). These molecules should therefore serve two purposes; first, as markers of the specific cell growth and differentiation state of the cell, or of the specific cell type; second, as a target for cell manipulation, as means to create new medications.

Cell state itself is a widely studied phenomenon with many components. Cultured stromal cells from the bone marrow re-create a hemopoietic inductive microenvironment in vitro (1-3); upon the formation of confluent adherent cell layers the stromal mesenchyme serves as a support for the lodging and long-term proliferation of hemopoietic stem cells (HSC). Similarly, these cells may promote engraftment of hemopoietic stem cells in vivo (4).

Cells derived from such stromal cultures were further identified as multipotent stromal cells (MSC) (5,6), or mesenchymal stem cells (7-9), since they give rise to mesodermal derivatives such as muscle, bone, cartilage and fat (10,11). Distinct mesenchymal cell types from bone marrow cultures have been isolated and propagated in the laboratory as permanent cell lines (MBA series) of osteoblasts, pre-adipocytes, fibroblasts, as well as cells with endothelial properties (12-14).

In recent years several reports demonstrated the existence of cells, such as multipotent adult progenitor cells (MAPC) (15,16), unrestricted somatic stem cells (USSC) (17), marrow-isolated adult multilineage inducible (MIAMI) cells (18), amniotic fluid-derived cells (AFS; 19), skin progenitor cells (SKP; 20) and stage specific embryonic antigen (SSEA)-1pos cells (21), that give rise either to cells of all embryo germ lines, or in other cases, cells that represent several unexpected lineages. The model that emerges from these studies is of an early mesenchymal cell that possesses pluripotent stem cell properties. In analogy with the hemopoietic system, it has been suggested that MSC, as well as the other related cell types, differentiate in a hierarchical manner by giving rise to cells of declining potency for self-renewal and decreasing range of cell types into which they are able to differentiate (22). In the hemopoietic system discrete types of committed progenitors were identified according to cell surface marker expression. These can be isolated to homogeneity and proven to be tri, bi or monopotent. By contrast, no restricted progenitors belonging to the MSC lineage have been identified with any certainty (reviewed in 23). The question whether differentiation of MSC obeys a hierarchical cascade of decreasing range of differentiation directions therefore remains open. It is further unclear where, within the differentiation cascade of MSC, the hemopoietic supportive capacity is positioned.

The ability of bone marrow mesenchyme to support hemopoiesis is thought to be dependent upon a multitude of factors including components of the extracellular matrix (ECM), cell surface constituents, soluble factors (including cytokines and small molecules) that make up the stem cell niche (3, 24-26). The minimal requirements for hemopoietic support have not, thus far, been conclusively defined. Furthermore, very little is known about the actual interactions between stem cells and their environment in terms of glycosylation patterns. A recent publication (27) indicated that ex vivo fucosylation of surface CD44 promote efficient adhesive interactions of MSC, leading to homing into the bone endosteal surface. Furthermore, the glycoprofile of MSC has been shown to determine their ability to support the ex vivo growth of HSC (28).

Hemopoietic stem cells are currently used in treatment of a wide range of pathological conditions in humans, and stem cell research is currently one of the most significant subjects in biology, in terms of prospective medical applications. Major drug companies such as GSK, Roche, AstraZeneca and Novartis are already entering the field, with the goal of obtaining sufficiently large quantities of stem cells to use for research purposes and a valuable new tool for use in drug discovery processes. Osiris Therapeutics is implanting so-called mesenchymal stem cells derived from bone marrow, in patients with heart disease and Chron's disease.

Developments in stem cell research are expected to change the face of medicine, providing new and novel ways to treat many currently incurable diseases, speeding up development of new drugs, eliminating unsafe medicines, creating better diagnostic tests. However, the number of hemopoietic stem cells currently available for transplant and research is limited.

It is considered that improvement of hemopoitic proliferation in culture, providing increased numbers of available stem cells, will greatly contribute to the fields of bone marrow transplantation and stem cell research.

### SUMMARY OF THE INVENTION

There is a need for a method and assay for detecting glycosylation patterns of stem cells, and their relationship to the state of the cells.

The present invention overcomes at least some of the deficiencies of the background art by providing such a method and assay, which in preferred embodiments are able to determine the state of a stem cell according to the glycosylation pattern for a plurality of different but correlated glycomarkers.

According to a preferred embodiment, the present invention provides a method of detecting the state of a stem cell, the method comprising contacting at least a portion of a stem cell with at least one saccharide-binding agent, determining binding of the saccharide-binding agent to the stem cell, determining the glycosylation pattern of the stem cell according to the binding of the saccharide-binding agent to the stem cell, and correlating the glycosylation pattern to the state of the stem cell.

According to some embodiments, at least two saccharide-binding agents are used in a single assay, preferably using whole stem cells (which may optionally be fixed), and/or non-whole stem cell material. Such non-whole stem cell material may optionally include a material selected from the group consisting of membrane protein extracts, homogenized cells, crude membrane mixture, crude cell mixture and/or any non-whole material derived from adding detergent and/or performing solubilization and/or extraction to cells. More preferably, the non-whole stem cell material is a crude cell mixture rather than a highly purified protein or group of proteins.

According to other embodiments, at least five saccharide-binding agents are used, preferably using whole stem cells (which may optionally be fixed), and/or non-whole cell material as described above.

As described in greater detail below, according to preferred embodiments of the present invention, the method and assay of the present invention are preferably performed in vitro, on a sample of stem cells and/or stem cell material.

The sample is preferably contacted with a glycomolecule detecting agent as described in greater detail below, such that at least a portion of the glycomolecules present in the sample are detected. A glycosylation fingerprint is then preferably determined for the sample, which is then preferably correlated with the state of a stem cell, with regard to differentiation.

Optionally and preferably, such a correlation is performed according to a comparison, such that if a glycosylation pattern matches a first category, then the sample correlates with a first cell stem state; alternatively if the glycosylation pattern matches a second category, then the sample correlates with a second stem cell state. More preferably, such a correlation may optionally feature a plurality of different categories relating to a plurality of different states, which most preferably fall along a continuum of stem cell functionality and/or behavior. The stem cell state is determined using the minimum number of data inputs required to differentiate between the different states. The first and second category may be, for example a differentiated and undifferentiated state, but may also optionally relate to differentiation to different types of cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. These include, but are not limited to, WO 00/68688 and WO 01/84147 (US20060194269, US20070092915, U.S. Pat. No. 7,056,678 and U.S. Pat. No. 7,132,251), WO 02/37106 (US20040132131), and WO 02/44714 (U.S. Pat. No. 7,079,955 and US20040153252). In the case of conflict, the present specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows the myelopoietic supportive capacity of mesenchymal cell populations. MEF supports long-term hemopoietic cultures starting from total bone marrow (A, B). Bar graphs show the total hemopoietic cell count (A) and myeloid progenitors count (GMCFU) per culture (B) at different time points. Comparison of the ability of different mesenchymal cell populations to support myelopoiesis (C, D). Hemopoietic cell count (C) and myeloid progenitors (D) were determined after 4 weeks in culture. E—early passages, L—late passages.

FIG. 2 shows an analysis of the ability of different mesenchymal populations to differentiate into mesodermal lineages. MBA-15, MSC, 14F1.1, MAPC-B, MEF-4 and MEF-5 were tested for their ability to differentiate in vitro into adiopgenic, osteogenic and chondrogenic lineages (A). Adipogenesis was indicated by accumulation of lipid droplets stained with oil red O. Osteogenesis was indicated by the increase of ALP expression in induced samples compared to control and by calcium mineralization as detected by alizarin red stain. Chondrogenesis was detected by accumulation of cartilaginous proteoglycans as detected by alcian blue staining. Quantification of adipogenic (B) and osteogenic (C) differentiation of mesenchymal cell populations. Cells were cultured in conditions favoring adipogenesis (B): insulin alone (dark gray bars), combination of insulin (Ins), IBMX and dexamethasone (DEX) (light gray bars) or without any inducers (black bars) or in conditions favoring osteogenesis (white bars) (C). After 4 weeks of culture, cells were stained with oil red O (B) or alizarin red (C). The dye was extracted and measured at 520 nm (B) or 415 nm (C) using spectrophotometer. Values were normalized to protein concentrations. E—early passages, L—late passages. Scale bar represents 100 μm.

FIG. 3 shows a comparison of the ability of MSC and 14F1.1 to support hemopoiesis after differentiation and the corresponding cytokines and growth factors expression. MSC were grown in normal (Control), adipoinductive (Adipo) or osteoinductive (Osteo) medium for 10 days and subjected to LTBMC conditions for 4 weeks. Hemopoietic cell counts (A) and myeloid colonies (B) were then determined. 14F1.1 were grown in normal (Control) or adipoinductive (Adipo) medium for two weeks and subjected to LTBMC conditions. Hemopoietic cell counts (C) and myeloid colonies (D) were determined. * p<0.017 and 0.00017 for Control-Osteo and Control-Adipo in A respectively; p<0.003 in B; p<0.004 in D; Semi-quantitative RT-PCR analysis of the expression of SCF, Flt3-L, M-CSF, IL-6, GM-CSF, LIF, IL-3 and G-CSF in differentiated samples of MSC and 14F1.1 after 4 weeks of LTBMC (E).

FIG. 4 shows cytokine and growth factors expression in MSC and 14F1.1 cells after differentiation and transfer to LTBMC conditions. Semi-quantitative RT-PCR analysis of the expression of SCF, Flt3-L, M-CSF, IL-6, GM-CSF, LIF, IL-3 and G-CSF in differentiated samples of MSC and 14F1.1 after 4 weeks of LTBMC.

FIG. 5 shows glycoprofiling of MSC and 3T3L1 after differentiation and the effect of glycosylation inhibitor on hemopoietic supportive capacity. Membrane extracts from MSC before (Control) and after adipogenic (Adipo) (A, B) or osteogenic (Osteo) (C, D) differentiation were analyzed on lectin microarrays. Total membrane proteins were extracted from cells as described in Materials and Methods and extracts were biotinylated and dialyzed prior to application to the lectin arrays. The fingerprints were detected using Cy3-labeled streptavidin. Profile obtained from three different lectins all specific to complex gycans is presented (1, 2, 3). MSC were treated with glycosylation inhibitor (0.4 mg/ml DMJ) and then analyzed on the lectin arrays. Profiles obtained from different lectins specific to complex (D), terminal sugars of complex glycans (E) and high mannose (F) are presented. To determine the effect of DMJ treatment on the hemopoieitc capacity of MSC, the cells were first incubated with the drug, then extensively washed and seeded with bone marrow cells. The formation of cobblestone areas after 2.5 weeks under LTBMC conditions is presented (G).

FIG. 6 shows a model of cell behavior. Mesenchymal cell populations behave according to phase-space model. Several directions of differentiation of the MSC may occur by direct derivation from the MSC itself, rather then from descendents of the MSC that progress through intermediate differentiation stages. MSC represent the same differentiation pattern observed for MEF-4 and MEF-1L but without adipogenic differentiation of the later; MEF-3L and MEF-7 were showing similar differentiation pattern of MSC but without exhibiting osteogenic differentiation. MBA-15 represents the same differentiation pattern observed for MBA-13, MAPC-A and MEF-1E; 14F1.1 represents the same differentiation pattern observed for MEF-6; MAPC-B represents the same differentiation pattern observed for C 3H₁₀T1/2, MEF-2, MEF-3E and MEF-5.

Table 1 shows the following (according to the scoring function):

Fat:

Oil red O OD/μg protein: 0-0.5−; 0.5-1+−; 1-2+; 2-3++; 3-4+++; 4-5++++; >5+++++;

ALP: +− Faint staining; + Regular staining; ++ Strong staining

Alizarin red OD/μg protein: 0-0.4−; 0.4-1+; 1-1.5++; 1.5-2+++; 2-2.5 ++++; >5+++++;

Cartilage:

Alcian Blue − no staining; +− Faint staining; + Regular staining; +++ Strong staining;

Supportive Stroma:

Total colonies (GMCFU) and cell counts −<50 colonies, 2×105 cells; +50-150 colonies, 2-4×105 cells; ++150-200 colonies, 4-6×105 cells; +++>200 colonies, >6×105 cells; ND.

### DESCRIPTION OF THE PREFERRED EMBODIMENTS

The present invention provides a method and assay for characterizing populations of stem cells according to their glycosylation pattern, preferably for distinguishing between cell populations.

Optionally, the state of the stem cell may be correlated to the glycosylation pattern by comparison to a known glycosylation pattern. Further optionally the glycosylation pattern may be computationally analyzed.

It has been suggested that epigenetic factors play a role in different biological processes (Feinberg, 2007), including the state of stem cells (Zipori, 2004).

The present inventors have surprisingly found that the glycosylation patterns of cells changes following their lineage specific differentiation, and contribute to differences in hemopoietic support.

As described in detail in the Examples section below, the present inventors performed glycoanalysis of protein membrane extracts from multipotent stromal cells, prior to and following induction of adipo- and osteogenic differentiation, using lectin microarrays. When comparing the binding patterns of the extracts from an undifferentiated MSC population with those of the differentiated cells, significant differences in signals were observed in a group of lectins that recognize complex N-linked glycans. These lectins recognize branching, at either of the two α-mannose residues of the tri-mannosyl core of N-linked complex glycans, and indicate the presence of either tri- or tetra-antennary structures. It was found that the level of antennarity, in the osteogenic cells derived from MSC, is higher than that of the undifferentiated MSC population. Conversely, following induction of adipogenic differentiation, the level of antennarity of complex glycans in the cells, is lower than that of the undifferentiated MSC population.

The glycosylation pattern of mesenchymal cells has been found to influence their capacity to support hemopoiesis in culture and seems to be associated with specific differentiation directions of the mesenchyme. Thus, sugar moieties on the surfaces of mesenchymal cells, which serve as a niche for HSC, are required by the stroma in order to correctly interact with the HSC. This was indicated by the finding that inhibition of glycosylation by 1-deoxymannojirimycin (DMJ), a known inhibitor of mannosidase I, prior to seeding the stroma with bone marrow cells resulted in reduced capacity of the stromal cells to support the formation of cobblestone areas (Morad et al, 2008). However, it is yet undetermined whether this phenomenon entails increased differentiation, or in contrast, suppression of differentiation and induction of HSC self-renewal. DMJ treated MSC are therefore examined in detail to establish whether the glycoprofile that results from DMJ treatment, is beneficial or disruptive for HSC self-renewal. Myelopoietic support was found to be dependent upon correct glycosylation of the stromal cells.

It is further considered important to determine the vitality of a specific glycosylation pattern, for the in vivo functioning of mesenchymal cells. Mouse and human mesenchymal cell populations, derived from independent sources, are analyzed to determine whether there is a constant glycosylation pattern, found in all cells that support HSC growth in culture. Cells conforming to this phenotype are further tested for their in vivo homing and migration to the bone marrow, to select for those that also contain glycoprofile that supports their migration and engraftment. If the glycoprofiles of homing and hemopoietic support coincide, this is a major indication for use of mesenchymal cell transplantation, as a therapy modality. Lack of correlation, would mean that cells should be selected according to a glycoprofile suitable for hemopoietic support, and then introduced directly into the bone marrow for proper functioning.

An extensive study of mouse bone marrow mesenchymal cell populations, propagated as continuous strains and cell lines, is performed. These cells show a divergent capacity to support HSC, and their corresponding glycoprofile is considered to be indicative of the glycosylation requirement of hemopoietic supportive stromal cells.

A series of 10-15 independent strains of MSC from normal mouse bone marrow, along with 5 primary human MSC stains is derived, the glycoprofile of the cell strains being determined at confluence. The cells are examined for their hemopoietic supportive ability.

The cell strains are further assayed for their in vivo homing capacity. Methods recently developed, for the labelling of cells and their follow up in real time, using modern imaging technologies, are employed to determine the in vivo localization of transplanted cells. The study comprises short term (1-3 weeks) analysis, using the above imaging; and long term (months) follow up, using biochemical analysis of green fluorescence protein (GFP), luciferase or Y chromosome analysis. In the case of human cell transplantation, chromosome analysis is the major tool. The study further includes a study of mesenchymal cells migration into tumor sites. Mesenchymal cells have been shown to either affect tumors by themselves, or otherwise serve to carry therapeutic molecules into the tumor site. The selection of mesenchymal cells having a high homing capacity to tumor sites and expressing a particular glycosylation pattern is examined.

The above studies provide an optimal pattern of glycosylation, for HSC maintenance and MSC homing. The present inventors further envisage developing highly effective MSC cells with a high capacity to support HSC and/or to effectively transplant in vivo, such as by modification, using siRNA technology to reduce the expression of specific glycosyltransferases thereby modifying the overall glycosylation profile.

The ability of stem cells to give rise to mature tissue cells may be harnessed for tissue regeneration, following injury or disease. The present inventors consider that identification of the exact glycoprofile needed for propagation of stem cells, which determines their migratory properties, and tendency to localize in tissues and engraft in them, will increase transplantability of these cells, by enabling design of populations capable of effective transplantation.

The present invention thus enables selection of optimal glycoanalysis patterns thereby determining stem cell functions, and providing better characterization of specific cell populations before and after differentiation. This method adds essential information to current methods of characterization using antibodies. The glycoanalysis technology of the present invention is rapid, easy to perform, cheap and therefore an ideal tool for QC applications, in the preparation and characterization of cells for cell therapy, and for the characterization of specific cell populations in stem cell research.

According to preferred embodiments of the present invention, an assay for detecting a glycosylation pattern of a stem cell may optionally and preferably be performed according to U.S. Pat. No. 7,056,678, owned in common with the present application, hereby incorporated by reference as if fully set forth herein, which describes methods and assays for detecting glycosylation of a stem cell. For example, this patent describes a method for the structural analysis of a saccharide, comprising: providing on a surface a plurality of essentially sequence-specific and/or site-specific binding agents; contacting the surface with a mixture of saccharides to be analyzed, for example an extract of glycomolecules from specific compartments of cells or tissue washing or otherwise removing unbound saccharide or saccharide fragments; adding to the surface obtained previously an essentially sequence- and/or site-specific marker, or a mixture of essentially sequence- and/or site-specific markers; acquiring one or more images of the markers that are bound to the surface; and deriving information related to the identity of the saccharide being analyzed from the image.

The surface on which the binding agents are provided may comprise, for example, a bead or an array.

Binding of the saccharide-binding markers may optionally be detected by acquiring images of the markers, and generating a map of recognition sites of the polysaccharide being analyzed, to derive partial sequence information relating to the polysaccharide.

The markers may optionally comprise chromogenic binding agents, such that images are provided which are colors that develop on the surface of the substrate. Alternatively, the markers may be labeled binding agents, such that images of the markers are provided according to a signal from the label. Images may be acquired, for example, by the use of optical filters, or by photographing and/or digitizing the images.

Additional methods and assays for determining a glycosylation pattern or “fingerprint” for a sample, such as for a cell for example, are also disclosed in US Patent Application No. 20050186645, also owned in common with the present application, which is hereby incorporated by reference as if fully set forth herein. This application describes a method for obtaining information about the carbohydrate content of a glycomolecule by adding a glycomolecule to a substrate to which is attached one or more saccharide-binding agents (also referred to herein as first saccharide-binding agents). The first saccharide-binding agents that have bound the glycomolecule are identified, and the resulting binding information is used to generate a fingerprint of the glycomolecule.

The essentially sequence- and/or site-specific binding agents of the present invention may comprise, for example, lectins (such as colored lectins, fluorescent lectins, biotin labeled lectins) or antibodies (such as fluorescent antibodies, biotin-labeled antibodies, or enzyme-labeled antibodies). The method or assay may be performed using at least five lectins, such as, for example, 5, 10, 15, 20, 25, 30, 35, 40, 45 or 50 lectins, although optionally any number of lectins may be used, for example from about 5 lectins to about 100 or more lectins.

For example, the method may optionally be performed with a set of 20-30 lectins with overlapping specificities, printed on a membrane-coated glass slide in replicates of 4-8, or alternatively in a range of concentrations that provide a dose-response for each printed lectin. A sample of intact glycoprotein is applied to the array, and its binding pattern is detected by either direct labeling of the glycoprotein using any fluorophore, or by using a fluorophore-labeled probe that is directed at either the protein moiety—an antibody for example, or a carbohydrate moiety—a lectin. The resulting fingerprints are highly characteristic of the glycosylation pattern of the sample. The large number of lectins, each with its specific recognition pattern, ensures high sensitivity of the fingerprint to changes in the glycosylation pattern. Many fluorescent labels such as FITC, Rhodamine, Cy3, Cy5, or any of the Alexa dyes can be used. These fluorescent labels and dye labels are collectively termed herein “chromogenic labels”. In addition, labeling can be effected using biotin-avidin systems known in the art and/or with any other suitable type of label. Glycomolecules may optionally be modified before being analyzed as described above.

The method and assay of the present invention may optionally be carried out on whole cells. Alternatively, the method and assay may be carried out on a cell preparation (non-whole cell material), such as, for example, a membrane protein extract, a homogenized cell, or a crude membrane mixture.

In embodiments which comprise the use of a whole cell, the cell is preferably first fixed. For example, the cells may be fixed in suspension of RPMI culture medium by adding 1% glutaraldehyde in Sorenson's buffer, pH 7.3 (Tousimis Research Corp., Rockville, Md.), and washing in Sorenson's buffer after 24-48 hours (as described for example in Sanders et al, A high-yield technique for preparing cells fixed in suspension for scanning electron microscopy, The Journal of Cell Biology, Volume 67, 1975, pages 476 480).

Alternatively, cells may be fixed by immersing in PBS/3.7% formaldehyde for 60 minutes at ambient temperature, after which the cells are washed in distilled water (as described for example in Nimrichter et al, Intact cell adhesion to glycan microarrays, Glycobiology, vol. 14, no. 2; pp. 197-203, 2004).

Of course any type of cell fixation process may optionally be performed which permits detection of binding of saccharide-binding agents to the cells.

The method of the present invention may optionally and preferably be performed in vitro.

The method and assay of the present invention may optionally and preferably be carried out using the Qproteome Glycoprofiling Kit (Qiagen USA). Lectins used in such kits have been chosen by analysis of a set of over 80 lectins, using a large dataset of carefully chosen, well-characterized glycoproteins, and a large set of enzymatically synthesized glycovariants of these proteins. The lectins on the array are grouped according to their monosaccharide specificities, in cases where possible; lectins in the group that is denoted “complex” do not bind monosaccharides, but bind complex N-linked glycans. The groups and differences between lectins within each group are detailed below.

### Complex

The lectins in this group recognize branching at either of the two α-mannose residues of the tri-mannosyl core of complex N-linked complex glycans. Some of the lectins of this group are sensitive to different antennae termini as they bind large parts of the glycan structure. The lectins denoted Complex(1) and Complex(4) have a preference for 2,6-branched structures; lectin Complex(3) has a preference for 2,4-branched structures, and lectin Complex(2) recognizes with similar affinity both structures.

### GlcNAc

The lectins in this group bind N-acetylglucosamine (GlcNAc) and its δ4-linked oligomers with an affinity that increases with chain length of the latter. The carbohydrate-specificity of both lectins in this group do not differ, yet differences in their binding patterns are observed and probably stem from the non-carbohydrate portion of the samples.

### Glc/Man

This group of lectins is a subgroup of the mannose binding lectins (see below), and are denoted Glc/Man binding lectins since they bind, in addition to mannose, also glucose. All of the lectins in this group bind to bi-antennary complex N-lined glycans with high affinity. In comparison to their affinity for bi-antennary structures, lectins Glc\Man(1) and (2) bind high mannose glycans with lower affinity, whereas lectin Glc\Man(3) will bind high mannose glycans with higher affinity.

### Mannose

This group consists of lectins that bind specifically to mannose. These lectins will bind high mannose structures and, with lower affinity, will recognize the core mannose of bi-antennary complex structures.

### Terminal GlcNAc

This lectin specifically recognizes terminal GlcNAc residues.

### Alpha Gal

These lectins bind terminal α-galactose (a-Gal). Lectin Alpha-Gal(1) binds both α-galactose and α-GalNAc (α-N-acetylgalactosamine) and may bind to both N and O-linked glycans. Lectin Alpha-Gal(3) binds mainly the Galili antigen (Gala1-3Gal) found on N-linked antennae.

### Beta Gal

These lectins specifically bind terminal (non-sialylated) β-galactose residues.

### Gal/GalNAc

These lectins are specific for terminal galactose and N-acetyl-galactoseamine residues. The different lectins within this group differ in their relative affinities for galactose and N-acetyl-galactoseamine.

Lectins (2) and (5) from this group bind almost exclusively Gal; lectins (1), (3) and (4) bind almost exclusively GalNAc. The relative affinities for GalNAc/Gal for the remaining lectins in the group are ranked: (8)>(7)>(6).

### Fucose

Lectins from this group bind fucose residues in various linkages.

Lectin Fucose(6) binds preferentially to 1-2-linked fucose; Lectin Fucose(8) binds preferentially to 1-3 and 1-6 lined fucose; Lectins Fucose(12) and (13) bind preferentially to Fucl-4GlcNAc (Lewis A antigens).

These lectins generally do not bind the core fucose of N-linked oligosaccharides on intact glycoproteins due to steric hindrance.

### Sialic Acid

The sialic acid lectins react with charged sialic acid residues. A secondary specificity for other acidic groups (such as sulfation) may also be observed for members of this group. Lectin Sialic Acid(1) recognized mainly 2-3-linked sialic acid; Lectin Sialic Acid(4) recognizes mainly 2-6-linked sialic acid.

The fingerprint itself provides valuable data for sample analysis. It is particularly useful for comparative analysis of several samples, to show differences in glycosylation.

Computational methods for analyzing the resultant glycosylation fingerprint data and for mapping glycosylation pattern(s) in the sample are disclosed for example in US Patent Application No. 20040153252, also owned in common with the present application, which is hereby incorporated by reference as if fully set forth herein. This application describes a method for computationally analyzing data from binding of a saccharide binding agent to a glycomolecule in the sample, such as lectin binding data for example, optionally with other types of binding data, to map the glycosylation patterns. A more detailed description of exemplary computational methods is provided below with regard to Example 2.

It should be understood that these examples for methods and assays for detecting glycosylation patterns in a sample, such as a cell for example, are provided for the purposes of discussion only and are not intended to be limiting in any way, as any other suitable method and/or assay could optionally be used with the present invention.

The principles and operation of the present invention may be better understood with reference to the drawings and the accompanying description, as well as the following examples.

### EXAMPLES

All the tissue culture, animal and molecular biology work are performed at the Weizmann Institute of Science, Rehovot, Israel.

### Example 1

Characterizing Stem Cell Populations

This Example relates to the characterization of cell populations through determining glycosylation patterns or fingerprints, herein for the comparison of differentiated cells to their undifferentiated progenitor stem cells. The methods described herein may optionally be used to compare any cell populations, including cells before and after exposure to certain treatments and so forth, but are preferentially described herein with regard to stem cells for the purpose of description only and without any intention of being limiting in any way.

The cells used in this Example were mouse embryonic stem cells (MES), which can be differentiated to neural cells as described below. The glycosylation pattern or “glycoprofile” of differentiated neural cells was compared to that of MES cells, which are not differentiated. This comparison demonstrated that the myelopoietic supportive capacity of mesenchymal stromal cells is not coupled to multipotency, but that it is influenced by lineage determination.

Materials and Methods

Animals: Mice were maintained under specific pathogen-free conditions. C57BL/6J and Balb/c mice were purchased from Harlan (Rehovot, Israel). TCR□-deficient mice (C57BL/6J-Tcrbtm1Mom) mice were obtained from the Jackson Laboratory (Bar Harbor, Me., USA) and propagated in the Weizmann Institute's animal housing facilities. All animal procedures were approved by the Weizmann Institute Animal Care Committee.

Cell culture: MBA-13, MBA-15, C3H10T1/2, 14F1.1 cell lines and mouse embryo fibroblasts (MEF) were grown in Dulbecco's Eagles medium (DMEM) (Gibco, Grand Island, N.Y., USA) supplemented with 10% heat inactivated fetal calf serum (FCS) (Biological Industries Ltd, Beit Haemek, Israel), selected according to its capacity to support the growth of the ABLS-8 pre B lymphoma cell line at the population doubling time of 10 hrs, supplement with 60 μg/ml penicillin, 100 μg/ml streptomycin and 50 μg/ml kanamycin. MEF were obtained from day14 gestation embryo fragments treated with trypsin-EDTA and were then propagated in accordance with procedures previously developed by the present inventors for the maintenance of non-tumorigenic mesenchyme (12,13) as follows: cells were examined frequently, following seeding, until reaching confluence. They were then passaged each time confluence was regained, rather than at fixed time intervals. Using this approach the cells did not undergo crisis and transformation and did not undergo senescence for over 15 passages.

MEF exhibited the cell surface antigen phenotype in which antigens most prominently expressed were ICAM1, MHCl and CXCR4 while the hemopoietic marker CD45 was not expressed at a detectable level. MEF-1, 4, 5 and 8 were derived from C57BL/6J mouse embryos. MEF-2 was derived from heterozygote TCR□−/+ mouse embryos. MEF-3, 6 and 7 were derived from TCR□-deficient mice. MSC were grown in murine MesenCult™ Basal Media supplemented with 20% murine mesenchymal supplement (StemCell Technologies Va, CA, USA), 60 μg/ml penicillin and 100 μg/ml streptomycin. MAPC were grown in MAPC medium consisting of 60% low-glucose DMEM (Invitrogen Life Technologies, Paisley, Scotland) and 40% MCDB-201 (Sigma, Rehovot, Israel), supplemented with 1× insulin-transferrinselenium (ITS), 1× linoleic acid-bovine serum albumin (BSA), 10-8M dexamethasone, 10-4M ascorbic acid 2-phosphate (all from Sigma), 60 μg/ml penicillin, 100 μg/ml streptomycin along with 2% FBS (HyClone Laboratories Logan, Utah), 1000 units/ml leukemia inhibitory factor (LIF) (Chemicon, Temecula, Calif.), 10 ng/ml epidermal growth factor (EGF) (Sigma), and 10 ng/ml platelet derived growth factor (PDGF)-BB (PeproTech/Cytolab, Rehovot, Israel) as described. All cells were incubated at 37° C. in a humidified atmosphere of 10% CO2.

MAPC derivation: Bone marrow (BM) was collected from the femur and tibia of 4 week old female C57BL/6J mice (n=7). BM mononuclear cells (BMMNC) were obtained by Ficoll separation and plated on fibronectin (Sigma) coated plates in MAPC expansion medium containing 2% FBS, EGF, PDGF-BB and LIF as described. After 6 weeks of expansion in culture, cells were depleted of CD45+/Ter119+ cells using micromagnetic beads (Miltenyi Biotec, Bergisch-Gladbach, Germany) according to the manufacturer's instructions. The depleted cells were then re-plated at 10 cells per well in 10 fibronectincoated 96 well plates and expanded as clones at densities of 2×102 cells/cm2.

MSC isolation and phenotypic characterization: BM cells were obtained from 7-8 week old C57BL/6J mice, pelleted, re-suspended in PBS and red blood cells lysis buffer (Sigma) for 5 min, and then subjected to an additional centrifugation. The cells were then seeded in 60 mm plates containing MSC medium. Half of the medium was replaced every 3 days and once a confluent layer was formed, the cells were removed using trypsin (0.05% EDTA, 0.25% trypsin) and reseeded. Cells were grown in culture for 4 weeks until a sufficient number of cells was obtained and then subjected to cell sorting.

Cell sorting: Primary BM cells were incubated with antibodies specific to CD45.2 Rphycoerythrin (RPE) (Southern Biotechnology Associates, Birmingham, Ala., USA) and CD11b/Mac1 fluorescein isothiocyanate (FITC) (Southern Biotechnology Associates, Birmingham, Ala., USA), for 1 hour and were then washed and suspended in PBS with 1% FCS. The cells were sorted using FACSVANTAGE cell sorter (FACSVANTAGE SE, Becton Dickinson Immunocytometry System, San Jose, Calif., USA). The double negative cell population was collected and seeded in MSC medium. Phenotypic characterization was performed as previously described by the present inventors (29).

Long-term bone marrow culture (LTBMC): Adherent cells were seeded in 6 wells plates (Falcon) and allowed to grow to confluency. BM cells from the femur and tibia of two 6-8 weeks old Balb/c mice were flushed out and 2×105 cells per well were seeded onto the confluent layers of adherent cells. Cultures were maintained in alpha-MEM (Gibco-BRL, Gaithersburg, Md., USA) medium supplemented with 20% horse serum

(StemCell Technologies) and 10⁻⁶M hydrocortisone hemisuccinate (Sigma) at 33° C. in a humidified atmosphere of 10% CO₂ for four weeks unless otherwise specified. Cultures were fed twice a week by replacing half of the medium with fresh medium. After four weeks, or on the specified day in culture, the non-adherent hemopoietic cells harvested, counted and subjected to granulocyte-macrophage colony-forming unit (GM-CFU) assay. Cells were seeded in methylcellulose semi-solid medium supplemented with 10 ng/ml interleukin (IL)-3, 10 ng/ml IL-6, 50 ng/ml stem cell factor (SCF) (all from PeproTech/Cytolab), and 3 units/ml erythropoietin (Epoetin alfa, Ortho-Biotech Janssen-Cilag, Baar, Switzerland). Cultures were maintained at 37° C., 10% CO₂ and scored, by morphology, on day 8.

Mesodermal lineage differentiation, detection and quantification: The basic medium used in all differentiation experiments was DMEM+10% FCS (HyClone Laboratories).

Adipogenesis: Cells were seeded at concentration to reach sub-confluency in a 24 wells plate. The following day, adipoinductive medium was added. Two conditions for adipogenesis were used: medium supplemented with 10 μg/ml insulin, 0.5 mM 3-isobutyl-1-methyl-xanthine (IBMX), and 1×10-6M dexamethasone (all from Sigma) or medium supplemented with 1.5 unit/ml human regular insulin (100 IU/ml Lilly HI0210). Cells were grown for four weeks with medium replaced twice weekly. Both adipoinductive media were used for the comparative study of mesenchymal cell populations. In all other experiments, adipogenic differentiation of MSC and 14F1.1 was carried out using 1.5 unit/ml human regular insulin only. Adipogenesis was detected by oil red O staining. For oil red O quantification, 4% IGEPAL CA 630 (Sigma) in isopropanol was added to each well. Light absorbance by the extracted dye was measured in 520 nm. Values were normalized to protein concentration.

Osteogenesis: Cells were seeded at concentration to reach sub-confluency in a 24 wells plate. The next day osteoinductive medium containing: 50 μg/ml L-ascorbic acid-2 phosphate (Sigma), 10 mM glycerol 2-phosphate di-sodium salt (Sigma), and 1×10-8M dexamethasone was added. The cells were grown for two (for alkaline phosphatase (ALP) staining) or four weeks (for alizarin red staining) with medium replaced twice a week. Osteogenic differentiation was detected by alizarin red staining. For alizarin red quantification, 0.5N HCl, 5% SDS was added to each well. Light absorbance by the extracted dye was measured in 415 nm. Values were normalized to protein concentration. ALP activity was detected by BCIP/NBT substrate chromogen system (Dakocytomation, Glostrup, Denmark) according to the manufacturer's instructions.

Chondrogenesis: Cells were grown in micro-mass culture supplemented with chondroinductive medium for four weeks. Cells at 2×105 per tube were centrifuged 5 min at 1200 g in 15 ml conical polyproylane tubes. Following centrifugation, the supernatant was gently removed and 1 ml of chondroinductive medium containing 0.1 mM L-ascorbic acid-2 phosphate, 10 ng/ml human TGF-beta1 (PeproTech/Cytolab), 1×10-7M dexamethasone was added. The tubes were incubated with the cap slightly loose, with medium replacement twice a week. After four weeks in culture the pellets were fixed with 4% PFA and embedded in 1.5% low melting agarose (Sigma) solution followed by paraffin embedding. Chondrogenesis was detected by alcian blue staining.

Statistical analysis: The Wilcoxon Ranksum Test using Matlab v.7.1 Statistical toolbox was applied to compare the mean of cell counts and GM-CFU colonies produced in LTBMC. Differences were considered statistically significant with p<0.05 for the comparison of 14F1.1 hemopoietic support after adipogenic differentiation (controladipo) or with p<0.025 when two analyses were performed (MSC control-adipo and control-osteo) to correct for multiple hypothesis with the Bonferroni correction. Semi-quantitative RT-PCR: Total RNA was extracted from MSC and 14F1.1 using Nucleo-Spin RNA II kit according to manufacturer's instructions (Macherey Nagel, Duren, Germany). Two micrograms of total RNA were reverse transcribed using Moloney murine leukemia virus reverse transcriptase (MMLV-RT) (Promega). PCR amplification of the cDNA was performed with ReddyMix PCR Master Mix (ABgene, Epson, United Kingdom). GAPDH expression was used as a control for loading and water was used as negative control.

Primer sequences used are as follows:

| | | |
|---|---|---|
| Gene | Sense | Anti-Sense |

| | | |
|---|---|---|
| SCF | GCTTGACTACTCTTCTGGAC | CTGCTGTCATTCCTAAGGGAG |
| | AA | |
| Flt3-L | CAGTCCCATCTCCTCCAACTT | AGCTGTGTGCAGGTGTCCTTC |
| M-CSF | AGTGAGGGATTTTTGACCCA | CTATACTGGCAGTTCCACCTGTCTG |
| | GGAAGCAAA | T |
| IL-6 | TGCACTTGCAGAAAACAATC | TGGTCTTGGTCCTTAGCC |
| GM- | CATTGTGGTCTACAGCCTCT | GGCAGTATGTCTGGTAGTAGC |
| CSF | C | |
| LIF | CATAATGAAGGTCTTGGCCG | TGCCATTGAGCTGTGCCAGTTG |
| | CA | |
| IL-3 | CCACCGTTTAACCAGAACGT | TCCACGGTTAGGAGAGACGGA |
| | TG | |
| G-CSF | TGCAGCAGACACAGTCCCTA | TGGCTGCCACTGTTTCTTTAGG |
| | A | |
| GAPDH | ACCACAGTCCATGCCATCAC | TCCACCACCCTGTTGCTGTA |

Glycoprofiling: Glycoanalysis of membrane protein extracts was performed using lectin microarrays.

Sample preparation: MSC were grown in 10 cm plates and induced to differentiate into adipocytes for two weeks and osteoblasts for three weeks with differentiation medium. Control plates were grown in MSC medium and harvested at confluence. Membrane proteins were extracted from the cells using the Qproteome Cell Compartment Kit (Qiagen, Hilden, Germany), as described in the user manual; briefly, extraction buffer CE1 was added to cells. This buffer selectively disrupts the plasma membrane without solubilizing it, thereby resulting in the release of cytosolic proteins. Lysates were centrifuged at 1000×g for 10 min at 4° C. The pellet, which contains intact plasma membranes and organelles, such as nuclei, mitochondria, and the endoplasmic reticulum (ER), was resuspended in extraction buffer CE2, which solubilizes all cellular membranes with the exception of the nuclear membrane. The suspension was centrifuged at 6000×g for 10 min at 4° C. The resulting supernatants, which primarily contain membrane proteins, were biotinylated using NHS-biotin (Pierce, Rockford, Ill.) at a ratio of 5:1 biotin molecules per protein molecule. Protein concentrations were measured using the BCA Protein Quantification kit (Pierce). The resulting samples were dialyzed in a Slide-A-Lyzer Mini Dialysis unit with a molecular weight cutoff of 7,000 D (Pierce) for 48 hours.

Glycoanalysis: Lectin microarrays as described above were blocked using 1% BSA (Sigma) and probed with each of the protein samples. The arrays were washed with PBS buffer containing 1 mM CaCl₂, 1 mM MgCl₂ and 0.1 mM MnCl₂. Detection of bound samples was performed using a second step of incubation with Cy3-conjugated streptavidin, and arrays were scanned with an Agilent microarray scanner. Results are shown for two out of three experiments performed for each differentiation condition.

In order to determine which differences in lectin signals are significant the two histograms of lectin signals to be compared were normalized using a robust regression algorithm. The particular algorithm used was a robust regression with MM estimates. This algorithm provides both the normalization factor between the two histograms, and an estimate of the similarity between them, which comes from the quality of the fit. This similarity was calculated using the weighted root mean square sum of the fit residuals (a), where the weights used are the factors assigned to each point by the robust regression calculation. The differences between signals in the two histograms were then calculated in terms of σ, and each difference larger than 2σ was considered significant.

Results: The results show a clear correlation between cell state and the glycosylation pattern(s) obtained there from in stem cells. In particular, as described in greater detail below, differences were seen in the glycosylation pattern of cells which underwent osteogenic differentiation as opposed to adipogenic differentiation: osteogenic differentiation was associated with an increase in the level of antennarity of N-linked glycans whereas adipogenic differentiation caused a decrease in antennarity of these glycans.

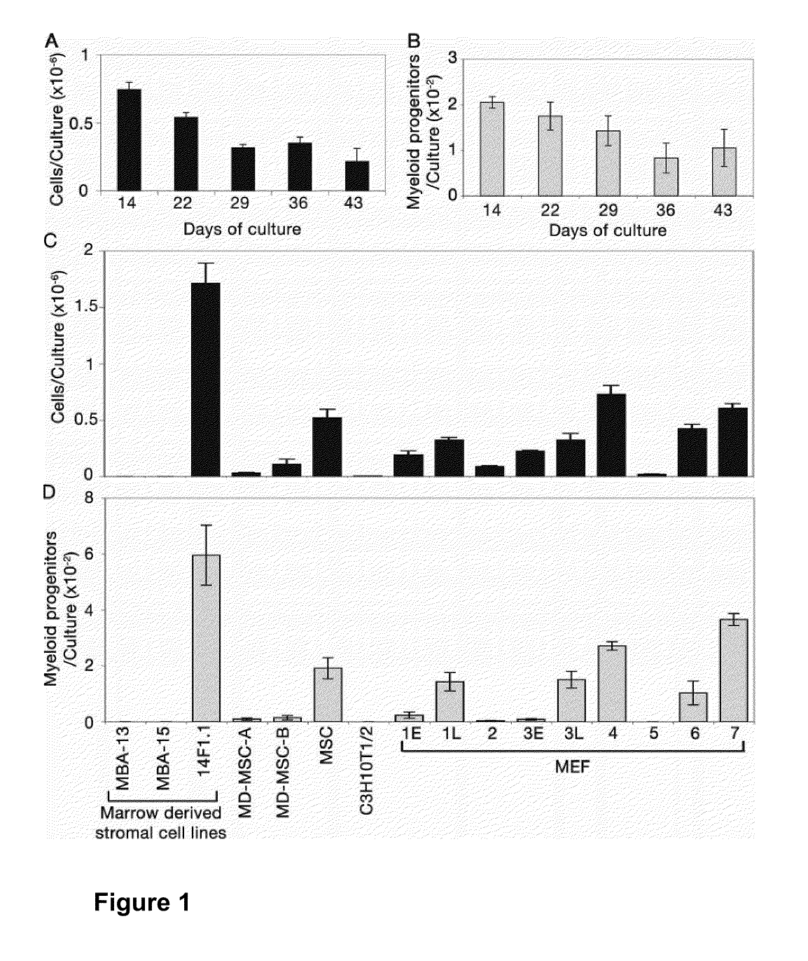

More specifically, FIGS. 1A and B show one example of bone marrow cultures supported by MEF. Hemopoietic cells including myeloid progenitors could be detected until the 43rd day of culture when the experiment was terminated. Similar experiments were performed with all other mesenchymal populations. The data are summarized as number of cells and myeloid progenitors per culture at four weeks of incubation (FIGS. 1C and D). The results represent one of at least three experiments performed for each cell population. Whereas the bone marrow derived 14F1.1 pre-adipogenic stromal cell line supported effectively long-term myelopoiesis, other stromal cell lines from bone marrow origin were devoid of this activity. Similarly, some MEF strains were supportive of myelopoiesis while others, derived using the same method and under the same conditions, were completely devoid of this property (FIGS. 1C and D). Some MEF retained a constant phenotype upon repeated passaging, with regard to their effect of long-term myelopoiesis. Others, like strain MEF-1 and MEF-3, were unstable i.e. these cells did not support myelopoiesis at early passages but did perform well at later passages. MSC showed a stable ability to support long-term myelopoiesis. Conversely, MAPC were ineffective in creating in vitro conditions appropriate for myelopoiesis (FIGS. 1C and D).

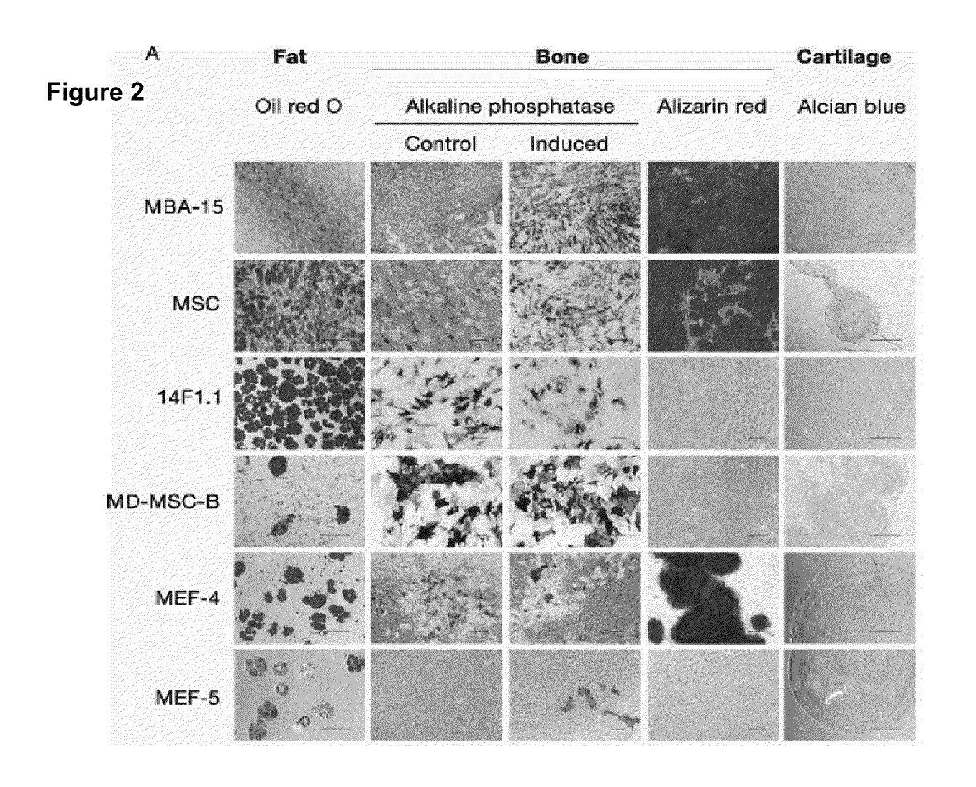

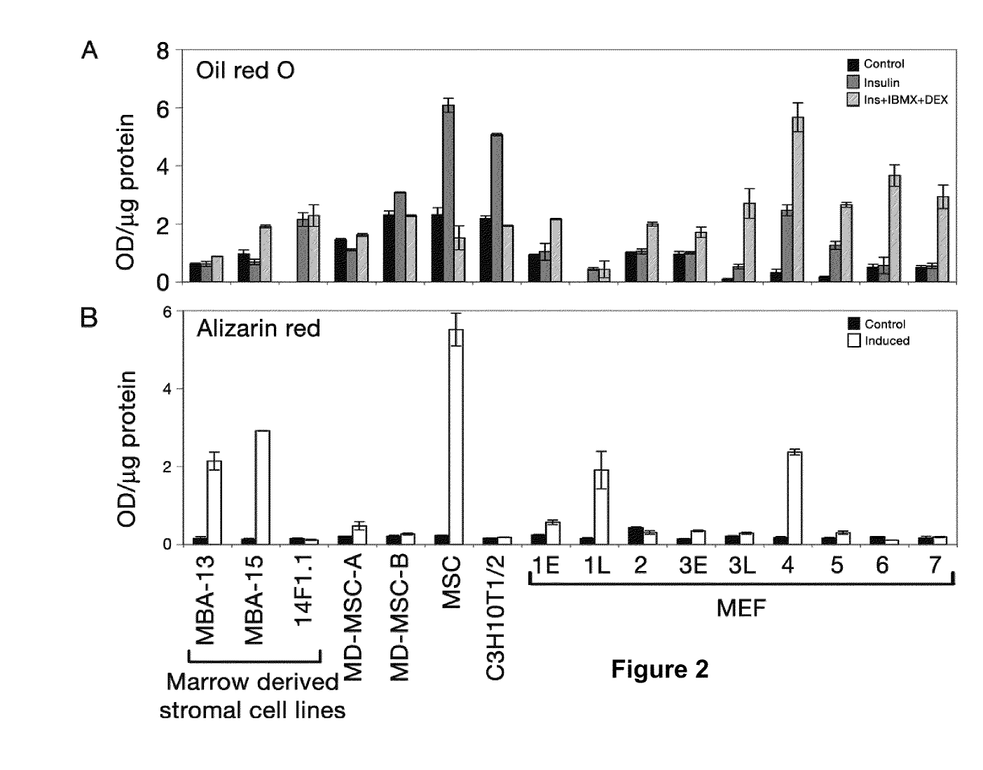

Mesenchymal cell populations were shown to vary in their capacity to differentiate into mesodermal derivatives. Cells within mesenchymal populations are capable of multilineage differentiation and are therefore designated as MSC. The cells were induced towards adipogenesis, osteogenesis and chondrogenesis. Examples of such induced differentiation are shown in FIG. 2A and quantitative determination of adipogenesis and osteogenesis, for the entire cell series is presented in FIGS. 2B and C, respectively. Clearly, different mesenchymal populations had a divergent capacity to differentiate into mesodermal lineages. Although to a different degree, adipogenic potential was very prevalent in the different cell populations whereas osteogenic potential was rare. Adipogenesis was most prominently observed in MSC, C3H10T1/2 and in some of the MEF strains, particularly MEF-4. MSC had the highest osteogenic capacity and additionally MBA-13 and MBA-15 stromal cell lines differentiated effectively into osteogenic cells, as did MEF-1L and MEF-4 (FIGS. 2B, C).

The myelopoietic supportive capacity of mesenchymal cells is uncoupled from their MSC multipotent phenotype A schematic summary and comparison between the cell's ability to differentiate into the three mesodermal lineages studied above and their corresponding capacity to support myelopoiesis is shown in Table 1. Whereas marrow derived stromal cell lines that are highly restricted to adipogenesis (14F1.1) had outstanding capacity to support myelopoiesis, others that differentiated into three mesodermal lineages were devoid of such capacity (MBA-15). Similarly, MEF-7 exhibited limited differentiation (adipogenic potential and some chondrogenic ability) but showed a strong myelopoietic supportive activity. MEF-4 supported myelopoiesis as effectively as MEF-7 but in contrast to the latter had a prominent differentiation capacity. Similarly, MSC that differentiate into all three lineages supported myelopoiesis effectively. Thus, the stem cell potential of a given mesenchymal population does not correspond to, and does not predict the ability of these cells to create conditions favorable for myelopoiesis.

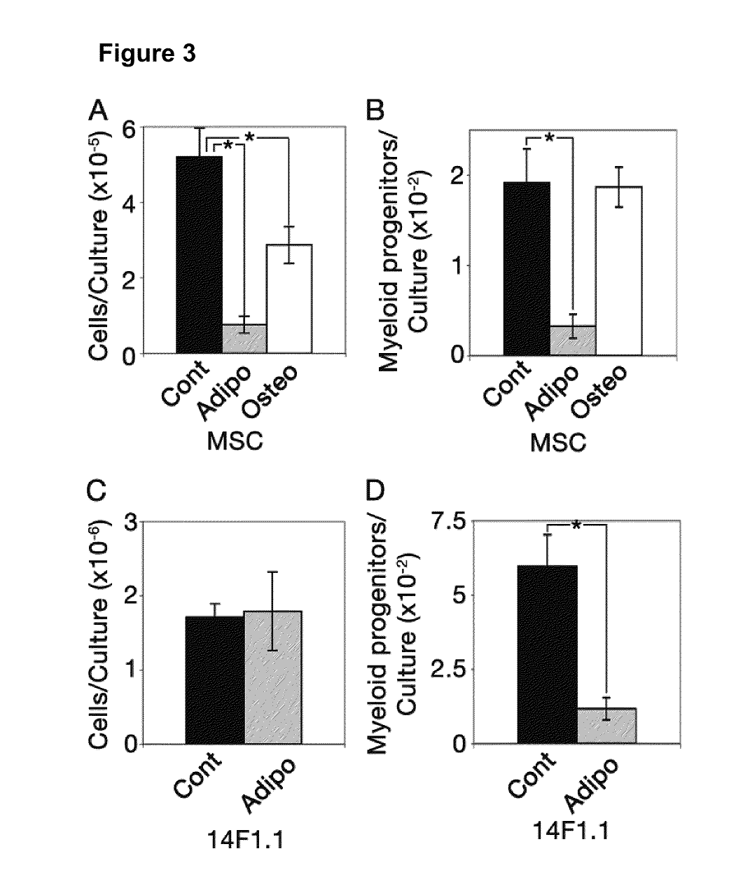

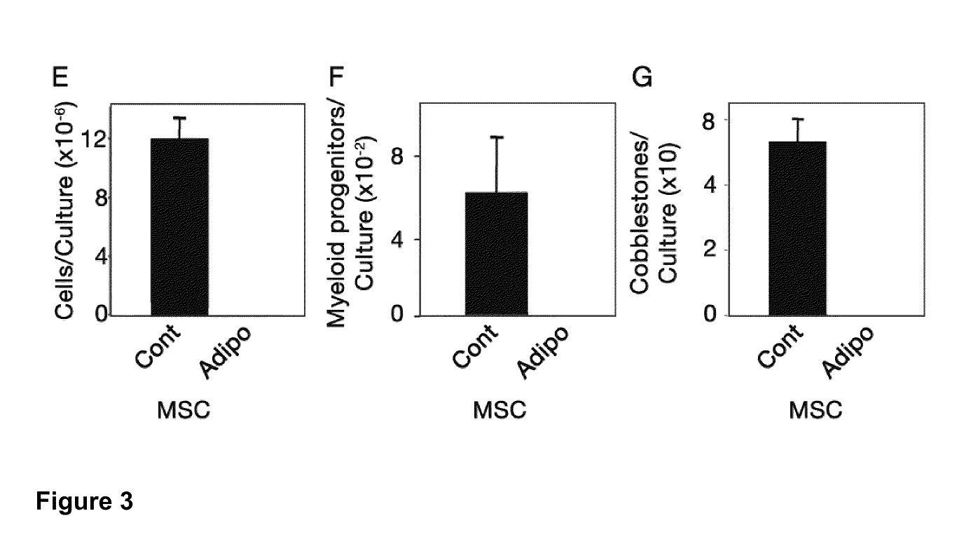

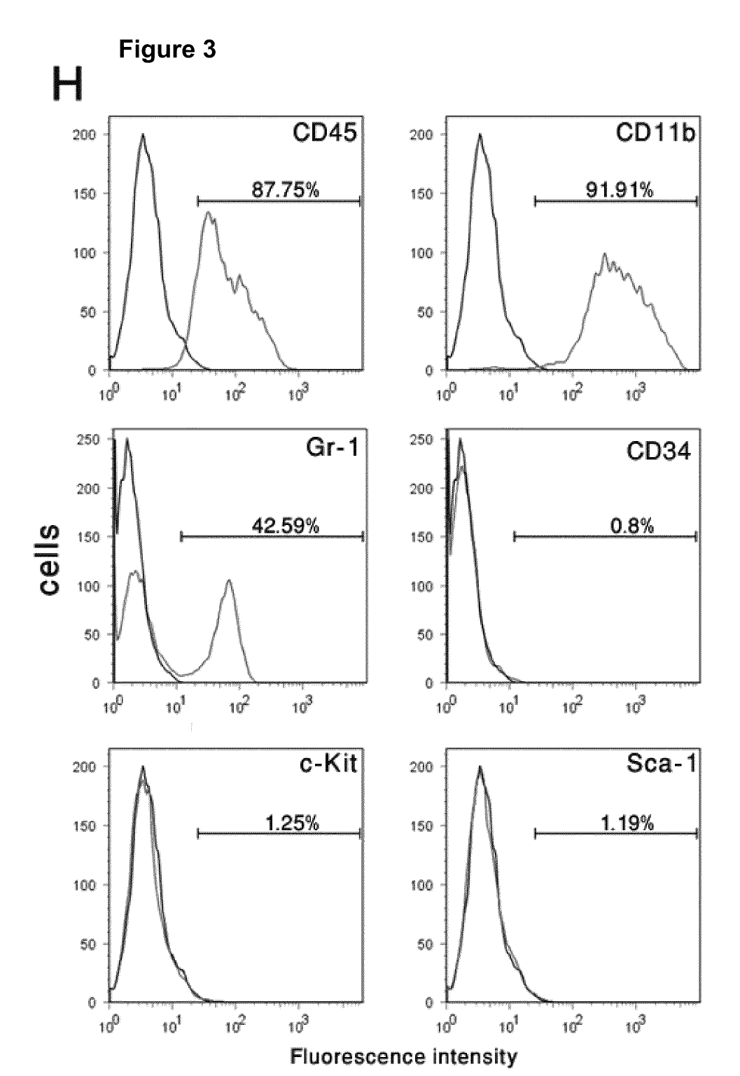

The differentiation of MSC into osteogenic lineage does not hamper myelopoietic supportive capacity whereas adipogenesis suppresses this function as shown by the following experiment. MSC were grown under osteogenic or adipogenic differentiation conditions for 10 days and were then examined for their myelopoietic supportive activity. Although osteogenic differentiation led to a 44.79% reduction in the hemopoietic cell yield, the incidence of myeloid progenitors recovered was unchanged (FIGS. 3A, B). In contrast, adipogenic differentiation reduced both the hemopoietic cell yield and the generation of myeloid progenitors by 85.41% and 83.09% respectively (FIGS. 3A, B). A similar experiment was then performed using the 14F1.1 pre-adipocyte cell clone. These cells are biased towards adipogenesis, as shown above. Upon induction of fat accumulation this stromal clone allowed the production of hemopoietic cells, however, as in the case of adipogenic MSC, the yield of myeloid progenitors was markedly reduced (FIGS. 3C, D).

Further analysis showed that upon adipogenic differentiation, MSC lost their capacity to support myelopoiesis. This was evidenced by complete inhibition of total cells (FIG. 3E), myeloid progenitors (FIG. 3F), and cobblestone area forming cells (FIG. 3G). By contrast, the control undifferentiated MSC sustained myeloid progenitors and supported cobblestone area formation. Flow cytometry analysis of cells maintained in the control MSC revealed that most of the cells expressed myeloid markers (91.1% CD11b) whereas a minor fraction was c-Kit+ (1.25%) and Sca-1+ (1.19%) (FIG. 3H).

Thus, whereas osteogenesis does not affect the yield of myeloid progenitors, adipogenesis causes drastic inhibition of progenitor cell accumulation in long-term bone marrow cultures. Table 1 summarizes the potential of the various cells studied to differentiate as compared to their ability, in the uninduced, non-differentiated state, to support myelopoisis. As can be seen, the mere potential of the cells to differentiate into a particular direction did not predict their myelopoietic supportive capacity. In contrast, the actual differentiation into specific mesodermal pathways seems to determine the ability of the cells to maintain active myelopoiesis.

Upon osteogenesis induction, an increase of ALP expression in induced samples was observed followed by calcium mineralization detected by alizarin red staining.

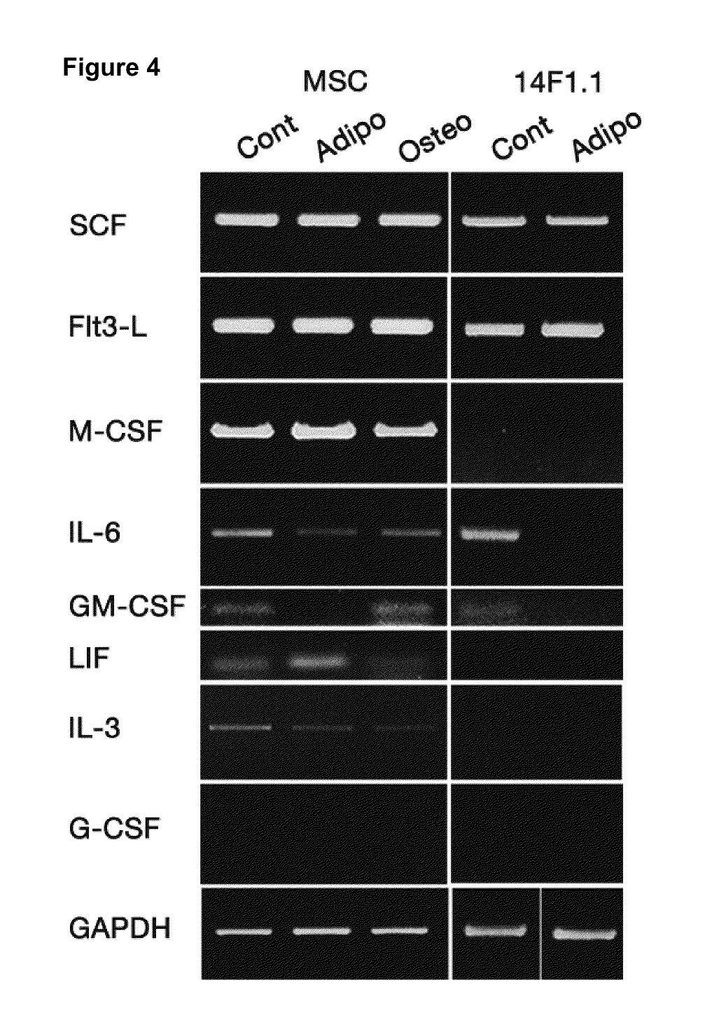

The induction of differentiation of MSC is associated with changed patterns in gene expression that may account for the reduced support of myelopoiesis post adipogenic differentiation. A group of cytokine genes was therefore selected based on their known contribution to hemopoiesis. IL-6 and GM-CSF expression, at the mRNA level, were reduced following adipogenic differentiation of both MSC and 14F1.1 cells. Conversely, Flt3L and SCF mRNA remained unchanged (FIG. 4). Interestingly, within the tested gene group osteogenesis did not significantly affect any of the hemopoietic cytokines, a finding that corresponds well to the limited effect of osteogenesis on myelopoietic supportive activity (FIGS. 3A and B).

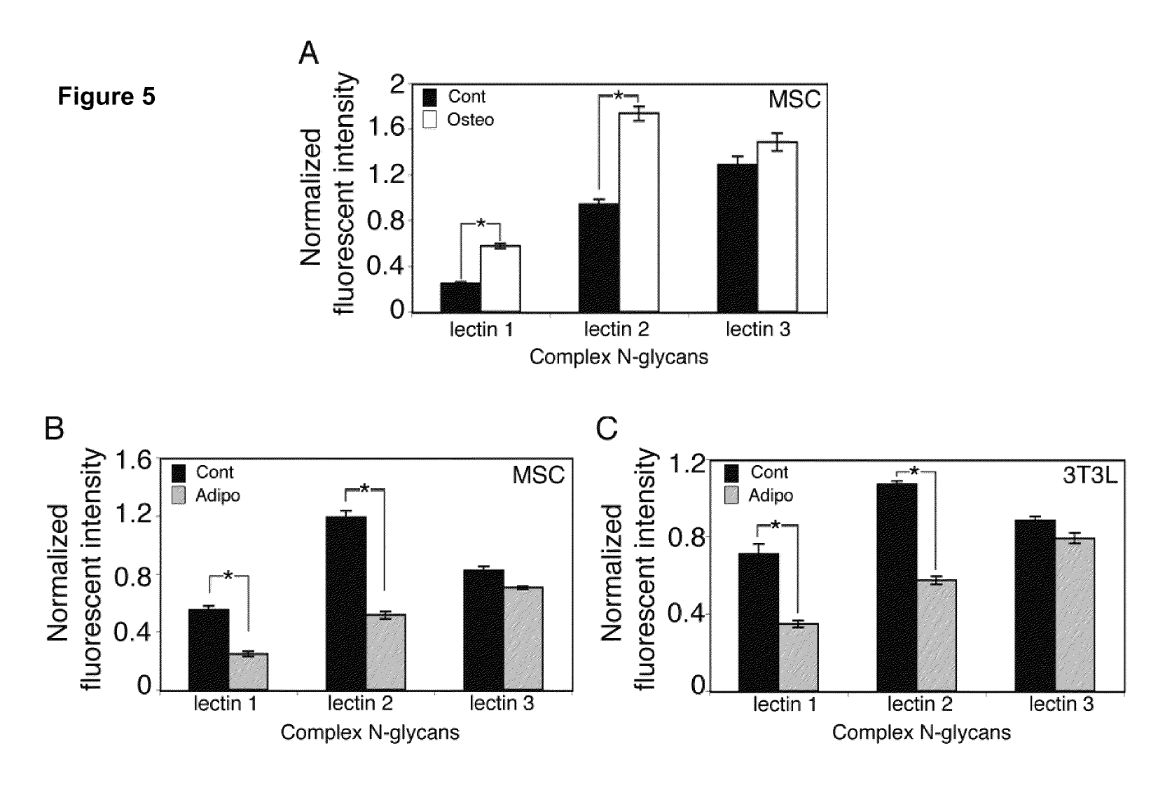

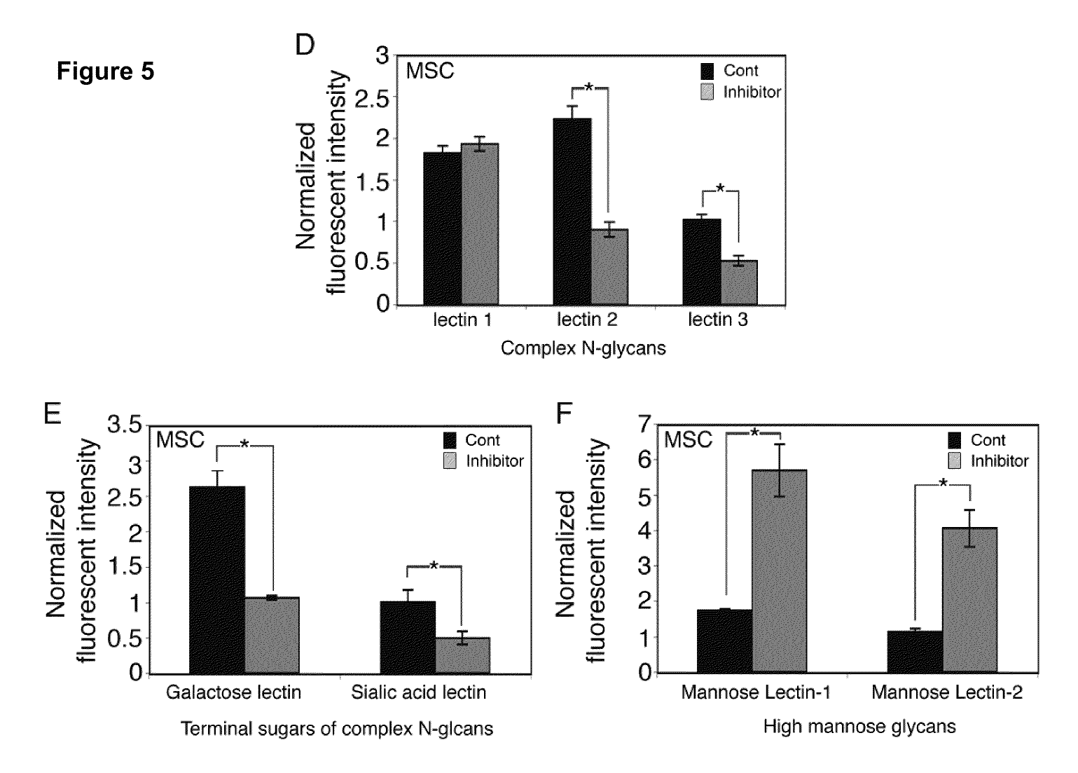

Glycoprofiling revealed differences between osteogenic versus adipogenic progeny of MSC. Glycoanalysis of protein membrane extracts from MSC, prior to and following induction of adipo- and osteogenic differentiation, was performed using lectin microarrays. When comparing the binding patterns of the extracts from undifferentiated MSC population to those of the differentiated cells, significant differences in signals were observed by a group of lectins that recognize complex N-linked glycans (FIGS. 5A and B). These lectins recognize branching at either of the two alpha-mannose residues of the tri-mannosyl core of N-linked complex glycans, and indicate the presence of either tri- or tetra-antennary structures. FIG. 5A depicts the binding of mesenchymal cell extracts to these lectins and demonstrates that the level of antennarity of the osteogenic cells is higher than that of the undifferentiated MSC population. FIG. 5B shows that following induction of adipogenic differentiation, the level of antennarity of the adipogenic cells is lower than that of the undifferentiated MSC population. A similar decrease in the level of antennarity of complex glycans is shown to accompany differentiation of NIH-3T3L1 fibroblasts into adipocytes (FIG. 5C).

Surprisingly the present results demonstrate that the myelopoietic supportive ability of stromal cells, whether from the bone marrow or from embryo origin, is not linked with multipotency; cell populations that possess multipotent capacity may or may not support myelopoiesis while others, lacking multipotency, may possess full myelopoietic supportive ability. However, upon differentiation, the ability of multipotent mesenchymal progenitors to support myelopoiesis is varied. Induction of these cells into osteogenic differentiation did not affect their ability to support myelopoiesis in long-term cultures. Conversely, adipogenesis resulted in reduced ability to support the maintenance of myeloid progenitor cells.

These results also support the concept that that glycoproteins contribute to the interactions of stromal cells and hemopietic progenitors and to the maintenance of the hemopoiesis in long-term culture. The modified glycosylation pattern that was observed following adipogenesis would appear to be associated with the change in myelopoietic support, without wishing to be limited by a single hypothesis.

Again without wishing to be limited by a single hypothesis, it appears that the differentiation of MSC and acquisition of mature phenotype, in terms of the capacity to support myelopoiesis, does not comply with a hierarchical cascade. The results described herein are more compatible with a phase-space model such as the hypothetical one shown in FIG. 5. This model is proposed as a possible interpretation of these results (again without wishing to be limited by a single hypothesis). It indicates that several directions of differentiation of the MSC may occur by direct derivation from the MSC itself, rather than from descendents of the MSC that progress through intermediate differentiation stages.

### Example 2

### Method for Glycoanalysis

According to some embodiments of the present invention, the results of one or more assays with saccharide binding agents are examined according to a method for glycoanalysis, which is optionally and preferably provided in the form of software (although it may alternatively may be provided as firmware or hardware), described herein as a “comparative interpretation module”. The comparative interpretation module is aimed at inferring changes in glycosylation between two samples based on significant lectin differences.

The module preferably comprises two sub-modules: a comparison module and an interpretation module. The comparison module normalizes the fingerprints and extracts the differences between them; the comparison module analyzes the list of differences in saccharide binding agent signals and deconvolutes them to provide differences in glycan epitopes. For the purpose of description only and without wishing to be limited, the method is described herein with regard to the binding behavior of lectins.

The algorithm used in this module preferably features at least one and more preferably a plurality of statistical classifiers, which have been extracted from a wide dataset of standards using machine-learning techniques. Each classifier maps a subset of lectin difference values onto a defined change in a single glycosylation epitope. The classifiers determine whether a change in a given epitope was detected, and if so, label it as an increase, decrease or (for some of the epitopes) a pattern change. Since the analyzed epitopes usually represent composite glycan structures, while the specificities of lectins are towards mono- or di-saccharides, the classifiers are based on deconvolution of signals from several lectins with overlapping and/or complementary specificities.

### Fingerprint Comparison Sub-Module

According to some embodiments, there is provided a fingerprint comparison sub-module. The input to the comparison sub-module is a pair of fingerprints, a reference and a target fingerprint. Initially, the fingerprints are normalized to enable a comparison of signals between the target and reference fingerprints. Following this normalization the fingerprints are compared and a list of differences is extracted.

Normalization is performed using a robust regression algorithm (the particular algorithm chosen is based on MM estimates). This algorithm extracts the largest subset of points, from both fingerprints, that produce the best possible fit. The algorithm provides both the best linear fit between the fingerprints, and an estimate of the similarity between the fingerprints, which comes from the quality of the fit. Also, the robust regression identifies the points that are outliers to the linear fit (outside the subset of the best fit), which correspond to the lectins that show appreciable changes between the fingerprints. These changes are quantified and transferred to the interpretation module.

In more details, standard regression models tend to break down when outliers exist. Robust regression methods attempt to find a fit that is independent of the existence of such outliers, by fitting a majority of the data. Regression with M estimates is an efficient, iterative method for removing outliers, provided that there are no leverage points (outliers at the extreme of the x-scale). Such leverage points cause the breakdown of the algorithm. In order to avoid this MM estimates are used. In this algorithm high-breakdown points are used to estimate the initial best-fit parameters, which are then improved iteratively by a minimization process, which in this case is optionally a Newton minimization (for example).

### Interpretation classifiers

According to some embodiments, there is provided one or more interpretation classifiers. The interpretation classifiers are mathematical functions that integrate various conditions for multiple lectin differences into boolean logical terms. In cases where a single lectin signal provides a reliable signal with a clear specificity, there is a single condition based on the difference level observed for this lectin that defines epitope changes. For other cases there may be several alternative criteria, each of which if met defines a change. In this way several different combinations of changes in fingerprint can lead to the same final verdict, which is in accordance with the fact that various changes can be manifested by a different lectin sets.

### Extraction and Calibration of Classifier

The above modules were tested with a benchmark of 878 fingerprint pairs that were successfully normalized in the fingerprint comparison module. These pairs were generated from 213 fingerprints from various cell lines, various biological systems, and enzymatically treated samples in which glycosylation patterns were altered in a controlled manner. Only pairs that were biologically comparable were considered for the normalization. For each pair, the expected result of at least one epitope was defined according to either (1) the particular treatment performed, (2) HPLC analyses, (3) ELISA experiments for fucose epitopes, or (4) literature reports. The benchmark was divided into nine partially overlapping training sets, each containing only pairs with a known change in a particular epitope. For each of these nine sets a set of control pairs, fingerprint pairs in which the examined change is expected not to occur, was compiled.

For each epitope, a logical rule was determined that best separates the dataset and its concomitant control. A statistical procedure was used to rank different Boolean functions that use different combinations of lectin differences, according to their ability separate the two sets. The procedure involved defining, for each epitope, a target function encompassing the sensitivity and specificity results that were obtained, and optimizing this target function on the dataset of fingerprint pairs described above. For each lectin, the minimal signal difference that is considered significant was automatically calibrated to achieve the partitioning of the highest quality. The automatically extracted rules were fine-tuned by careful manual analysis, based on the known specificities of the printed lectins. This analysis resulted in various heuristic rules that either enhance performance or deal with contradicting evidence.

Examples of calibrated rules and their concomitant verdict are listed in Table 2:

Examples of Comparative Interpretation Rules

TABLE 2

| | |
|---|---|
| Rule* | Verdict |

| | |
|---|---|
| (d(Alpha Gal(1)) > 4 and d(Alpha Gal(3)) ≧ 0) | Increase in α-Gal |
| or | |
| (d(Alpha Gal(1)) > 4 and ((d(Beta Gal (2)) < 0 | |
| and d(Gal\GalNAc (8)) ≦− 1) or (d(Beta Gal | |
| (2)) ≦− 1 and d(Gal\GalNAc (8)) < 0) or | |
| (d(Beta Gal (2)) ≦− 6 and d(Beta Gal (1)) < 0)) | |
| or (d(Alpha Gal(3) > 5) and d(Alpha Gal(1)) ≧ 0)) | |
| d(Sialic Acid (4)) > 2 and d(Beta Gal (2)) <− 3 | Increase in sialic acid |

*d(XXX) = difference in lectin XXX as measured by comparison of test sample to reference sample.

### Results

The performance was calculated for each epitope independently using the appropriate dataset, in which the expected interpretation of this epitope is known. The datasets are divided into a set of fingerprint pairs that are expected to show a change in the examined epitope, and a control set, in which the examined change is expected not to occur. Table 3 summarizes the performance of the algorithms on the entire benchmark. The sensitivity errors are broken down into 1-level and 2-level errors, denoting if the change was not detected (1-level), or detected in the wrong direction (2-levels). This breakdown is not applicable to the specificity analysis, since false positive detection of changes can only be a 1-level error.

TABLE 3
performance summary

| | Sensitivity analysis | | | Specificity |
|---|---|---|---|---|
| | | 1 level | 2 level | analysis |
| | Sensitivity | error | error | Specificity |
| N-linked glycans | | | | |
| antennarity of complex | 71% | 29% | 0% | 89% |
| glycans | | | | |
| oligomannose epitopes | 68% | 32% | 0% | 89% |
| O-linked glycans | | | | |
| global pattern change | 50% | NA* | NA | 72% |
| Terminal sugars | | | | |
| sialic acid | 74% | 26% | 0% | 81% |
| beta-galactose | 79% | 21% | 0% | 88% |
| alpha-galactose | 79% | 18% | 2% | 80% |
| GlcNAc | 100% | 0% | 0% | 95% |
| fucose-alpha(1-2) | 74% | 23% | 3% | 95% |
| fucose (other) | 54% | 45% | 1% | 96% |

*Since the function for O-linked glycans identifies a change in global pattern, the breakdown of errors is not applicable.

These results clearly indicate that the method and modules described above are sufficient to be accurate discriminators between different types of binding results.

### Example 3

### Clinical Applications of Determining the Glycosylation Pattern of Stem Cells

This Example relates to uses of the present invention for determining the glycosylation pattern of stem cells, particularly with regard to clinical applications. Human stem cells have been proposed for use (and/or are already in use) as transplants to patients who are in need of treatment for various diseases and injuries, including but not limited to Parkinson's disease, heart disease, blood cancers (such as leukemia), non-cancerous blood diseases (such as aplastic anemia), spinal cord injuries, brain damage and the like. It is important to monitor the state of such stem cells in vitro to make certain that the correct state is maintained before transplantation, whether to make certain that the stem cells remain undifferentiated and/or to make certain that the stem cells differentiate correctly to the desired differentiated cell type. Other uses include determining that the stem cells are differentiating according to the correct pathway(s) and/or maintaining control of differentiation.

For these uses, a human stem cell population is preferably assayed as described above, and the glycosylation pattern is determined. A determination may optionally then be made as to whether the cells are to be treated with a one or more treatments and/or whether some other change (addition and/or removal of one or more materials for example, and/or a change to an environmental condition) is preferably to be made according to the results, for example. Alternatively or additionally, the human stem cells are then used for transplantation or rejected for transplantation, according to the results, as another example.

### Example 4

### Effect of DMJ Treatment on HSC Self-Renewal

The initial steps in N-glycan synthesis involves synthesis of a precursor oligosaccharide, which is then stepwise processed by several enzymes, including mannosidase I, to allow synthesis of complex N-linked glycans. Deoxymannojirimycin (DMJ), a known inhibitor of mannosiase I, blocks the enzyme and therefore inhibits conversion of high mannose to complex chains. As a result, treatment with DMJ leads to synthesis of glycoproteins with increased levels of high mannose glycans and less complex N-linked glycans.

To test the possible contribution of stroma glycosylation to the support of hemopoiesis, glycosylation in MSC was inhibited with DMJ (Sigma), and the effects on hemopoietic supportive capacity were examined. MSC were analyzed on lectin arrays.

MSC were incubated for 3 days in the absence or presence of 0.4 mg/ml DMJ. Total membrane proteins were extracted from cells and applied to the lectin arrays. Profiles obtained from different lectins specific to complex, terminal sugars of complex glycans and high mannose were detected. MSC were similarly treated with or without DMJ for 2 days, washed 4 times with PBS. Cultures were then seeded with BM cells and subjected to LTBMC conditions. Two and a half weeks later, the number of cobblestones was scored.

### Results

DMJ treatment caused a decrease in antennarity (FIG. 5D) that was also accompanied by decrease in sugars that are found on antennae termini such as beta galactose and sialic acid (FIG. 5E). Moreover, signals from all oligomannose binding lectins were significantly increased, suggesting that the glycoproteins contain increased amounts of high mannose glycans in comparison to the non-treated cells (FIG. 5F). When DMJ treated MSC were compared to controls for their capacity to support cobblestone area formation, following removal of the inhibitor, 42% reduction was observed (FIG. 5G). To test whether the reduction in the number of cobblestones was due to a direct effect of DMJ on hemopoietic cells, bone marrow cells were subjected to GM-CFU assay in the presence of DMJ (3×10⁻³M, 3×10⁻⁵M and 3×10⁻⁷M). No statistically significant differences between cultures grown in the absence or in the presence of these low concentrations of DMJ were observed (data not shown).

### Example 5

### Selection of MSC Having High Capacity to Support HSC and/or High Transplantability

A series of mouse and human MSC strains is derived, an analysis of the glycoprofiles of the cell populations is undertaken, and the capacity of the cell to support HSC is tested. The glycosylation requirement of HSC-supportive stromal cells is thereby determined, to enable selection of MSC having a superior glycosylation profile for this purpose.

Glycoanalysis. Glycoanalysis is reported previously (Morad et al., 2008). Lectin microarrays, provided by Procognia Ltd., (Ashdod, Israel) are blocked using 1% BSA (Sigma) and probed with each of the protein samples. The arrays are washed with PBS buffer containing 1 mM CaCl₂, 1 mM MgCl₂ and 0.1 mM MnCl₂. Detection of bound samples is performed using a second step of incubation with Cy3-conjugated streptavidin, and the resulting arrays scanned with an Agilent microarray scanner. In order to determine which differences in lectin signals are significant, the two histograms of lectin signals to be compared are normalized using a robust regression algorithm with MM estimates (Marazzi, 1993). This algorithm provides both the normalization factor between the two histograms, and an estimate of the similarity between them, which comes from the quality of the fit. This similarity is calculated using the weighted root mean square sum of the fit residuals (a), where the weights used are the factors assigned to each point by the robust regression calculation. The differences between signals in the two histograms are then calculated in terms of a, and each difference larger than 2σ is considered significant.
In vitro and in vivo analysis of human HSC: Human CD34+ cells are enriched using magnetic bead separation kit to a purity of about 90%. The quality of the population is examined by flow cytometry using antiCD34 and antiCD38 antibodies. Myeloid progenitor cells are examined using semisolid clutres. 2×10⁵ cells are seeded in methylcellulose cultures supplemented with FCS and human plasma (15% each), Stem cell factor, interleukin-3, granulocyte macrophage colony stimulating factor, and erythropoietin. Colonies are counted at day 14 culture. For analysis of HSC repopulating capacity in vivo, 8 week old NOD/SCID mice are irradiated at 375 cGay and injected with human CD34+ cells at 2×10⁵ per mouse. At 3 month following transplantation, human cells are enumerated in the mouse bone marrow by flow cytometry using antibodies to human HSC as well as by Southern blot analysis for human DNA using human-specific a satellite probe as previously reported (Peled et al. Science, 283: 845-848, 1999).

Cell migration and homing: The study of cell migration lags such that glycoprofiles of the cells studied is known when their corresponding migration is examined. Cell migration and homing are performed using cell populations labeled with the fluorescent agent 1,1′-dioctadecyl-3,3,3′,3′-tetramethylindotricarbocyanine iodide. The labeling is performed by a short in vitro incubation of the cells with the dye. Such a procedure does not cause any extensive modification of the tested cell that often results from genetic labeling. It is also short and efficient and can be applied to a large number of samples. Preliminary experiments show that this strategy yields cells that can be followed in vivo for up to 7 days, by use of live imaging. The technical details of these experiments are as follows: Cells are labeled with 1,1′-dioctadecyl-3,3,3′,3′-tetramethylindotricarbocyanine iodide (DiR, Invitrogen). For labeling, cells at, 1-3.5×10⁷, are incubated in 10 ml PBS containing 3.5 μg/ml dye and 0.5% ethanol at 37° C. for 40 minutes with two subsequent washes with PBS. Afterwards, 0.5, or 1 or 2×10⁶ cells are injected i.v. Imaging is done under 2% isoflurane anesthesia, using the Xenogen In Vivo Imaging System (IVIS 100, Weizmann Institute of Science, Rehovot, Israel), or IVIS Spectrum (Caliper Life Sciences Inc, Hopkinton, Mass.).

### Example 6

### Genetic Modification of MSC to Increase Support of HSC and/or Transplantability In Vitro

Standard siRNA technology is used to reduce expression of various glycosyltransferases. The cells that show increased activity, in either stem cell support or otherwise in migration, indicate that the specific down-regulated gene should be knocked out, in order to provide a better functioning cell. If on the other hand the down-regulation of a specific gene causes reduced activity, overexpression is attempted. The methods for assaying for hemopoietic support as well as cell migration are as in Example 5.

### Example 7

### Selection of MSC Having Superior In Vivo Homing and Engraftment in the Bone Marrow and in Tumor Sites

Cells exhibiting glyosylation pattern that are found to be superior in terms of hemopoietic support and/or migration are labeled as in Example 5 and injected into tumor bearing NOD/SCID animals. Cell targeting to the tumor is analyzed by real-time imaging as above.

### Example 8

### Testing New Lectins and Antibodies Directed Against Glycan Epitopes

Several glycan epitopes, which have been found mainly on cell surface proteins, have been shown to be related to changes in several biological processes. These glycan epitopes include poly sialic acid and core fucose, which are unregulated in several types of cancer.

New lectins recognizing core fucose and poly-sialic acid (PSA), as well as an anti-PSA antibody are tested. Also, mammalian lectins (from human origin) are applied to improve sensitivity for the Lewis-X and Lewis-A antigen.

### Discussion

Cultured stroma cells are capable of creating a substratum for the maintenance of long-term hemopoiesis. Hemopoietic stem cells adhere to cultured stroma, form cobblestone areas underneath the adherent stromal cell layer, remain proliferating, and eventually give rise to hemopoietic progenitors and subsequently to mature cells that depart from the adherent layer and accumulate in suspension. This in vitro culture demonstrates the formation ex vivo of a hemopoietic niche. It should be noted though that the cellular structure that forms in vitro is highly complex and extreme diversity in cell phenotypes and interactions have been reported to occur in long-term bone marrow cultures. Apart from the niche forming ability of bone marrow stroma, at least a fraction of cells within such stroma possess multipotency i.e. can give rise to a variety of mesodermal cell types.

The present inventors examined whether the ability to support myelopoieisis is linked with the multipotency of stromal cells. Specifically, the possibility that the supportive activity of the stroma is a specific MSC marker was tested. It had been suggested that a central component of the stem cell niche is the osteoblast. This cell can be generated through the differentiation of MSC.

The results shown herein indicate that mesenchymal cells may or may not possess myelopoietic supportive capacity. In addition, the ability to support myelopoieis may be exhibited at the undifferentiated MSC stage, but can also be a property of the fully differentiated progeny of the MSC i.e. osteogenic cells that deposit bone mineral in culture. This does not mean that cells should differentiate into osteoblasts and osteocytes in order to support hemopoiesis. Indeed, pre-adipogenic cells, such as the 14F1.1 cell line, or MSC, support myelopoieis well without showing any bone forming functions. 12F1.1 pre-adipocytes are further adipogenic lineage restricted and do not have an osteogenic option at all. The use of such cell lines is limited by the fact that due to their immortalization, their responses may not represent those of primary cells. However, the present data confirm the similarity between such cell lines and MSC populations.

MSC differentiation has been suggested to be organized in a hierarchical cascade. In such a hierarchical model, one would expect the cells to acquire the hemopoietic supportive capacity upon induction of differentiation and loss of stemness. Yet, it appears that the hemopoietic support property is either associated with the stem cell itself, or alternatively with a differentiated progeny, such as the pre-adipocyte or the osteocyte, to mention two examples. The property therefore seems to exist throughout the differentiation cascade rather than emerge at any particular stage. It is therefore not the case that MSC differentiation into hemopoietic supportive stroma. They may either serve this function, as they are (i.e. while maintaining their undifferentiated stemness), or they may also fully differentiate and still maintain this function. This latter event occurs during osteogeneis while adipogenesis seems to interfere, at least partially, with the capacity to support myelopoiesis.

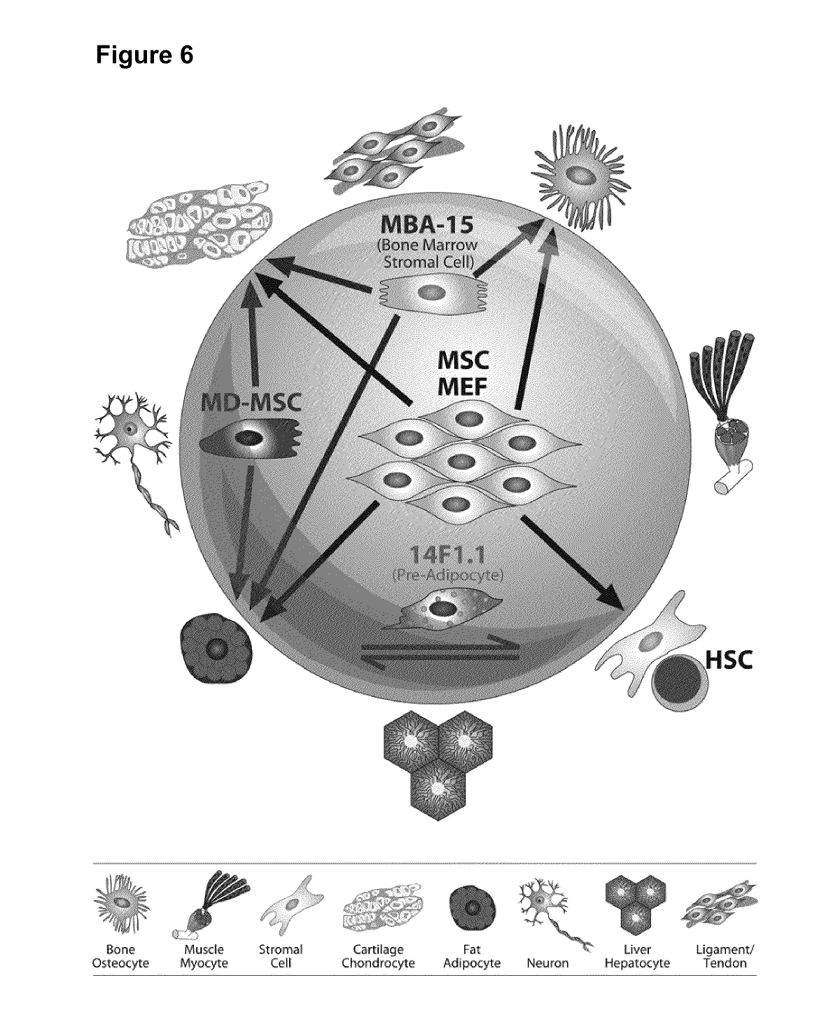

It is concluded that the differentiation of MSC and acquisition of mature phenotype, in terms of the capacity to support myelopoiesis, does not comply with a hierarchical cascade. The present results are more compatible with a phase-space model, such as the hypothetical one shown in FIG. 6. This model is proposed as a possible interpretation of the results presented in this study, and is also based on previous observations related to the plastic nature of mesenchymal cells. It indicates that several directions of differentiation of the MSC may occur by direct derivation from the MSC itself, rather than from descendents of the MSC that progress through intermediate differentiation stages. Furthermore, the model proposes possible reversibility of differentiated phenotypes.

Previous studies by the present inventors have shown that the MBA-14 cell line, that constituted a mixture of fibroblast-like cells and macrophages, could be separated into two distinct populations, 14F and 14M. The former became strongly fat laden upon separation from the latter. The pre-adipogenic cell supports hemopoiesis and performs better when in the pre-adipogenic rather than in the highly adipose laden state. Upon re-addition of the 14M macrophages to the 14F fat laden cells, the latter regained the fibroblast appearance. These studies, that indicate possible de-differentiation of adipogenic cells, are supported by a recent publication showing that adipose tissue cells can convert into fibroblastic phenotype and, upon this conversion, gain multipotent differentiation features.

One more issue raised by the present inventors is that the myelopoietic supportive function of mesenchymal cell populations may be gained by populations that do not possess this function a priori. Whether this is due to the selection of rare clones, which take over during culture, or to a generalized phenotypic change, is at the moment unclear. This contention requires further elucidation using single clone analysis.

The finding that osteogenic differentiation does not interfere with the capacity of MSC to support myelopoieis goes along with the fact that osteoblasts contribute to the stem cell niche in vivo. By contrast, adipogenic differentiation of human mesenchymal cells has been shown to reduce the capacity of these human cells to support the proliferation of cord blood CD34+CD38 progenitors. Similarly, it is shown herein that adipocyte differentiation of mouse MSC results in reduced maintenance of myeloid progenitors. The mechanism seems to involve reduction in expression of IL-6 and GM-CSF.

An additional occurrence associated specifically with adipocyte differentiation was a change in glycosylation pattern. It has been previously shown that free saccharides interfere with the interactions of hemopoietic progenitor cells and the stroma. Subsequent studies with bound sugar moieties substantiated these findings. It is therefore implied that glycoproteins contribute to the interactions of stromal cells and hemopoietic progenitors, and to the maintenance of the hemopoieis in long-term culture. It is thus proposed that the modified glycosylation pattern observed following adipogenesis is associated with the change in meyleopoietic support. Indeed, the use of an inhibitor of glycosylation (DMJ) lead to reduced capacity of treated MSC to support the formation of cobblestone areas in co-cultures with bone marrow cells. Due to the reversible nature of the DMJ mediated inhibition, it is likely that the effect observed has to do with the initial immediate interaction, probably the adhesion, of hemopoietic progenitor cells within the stroma. Glycosylated moieties present in stromal cells seem therefore to contribute to the interactions between the stroma and hemopoietic progenitor cells. Such interactions may be of importance for the formation of hemopoietic stem cell niches in vivo.

While the invention has been described with respect to a limited number of embodiments, it will be appreciated that many variations, modifications and other applications of the invention may be made.

### REFERENCES

1. Friedenstein A J, Chailakhyan R K, Latsinik N V, Panasyuk A F, Keiliss-Borok I V. Stromal cells responsible for transferring the microenvironment of the hemopoietic tissues. Cloning in vitro and retransplantation in vivo. Transplantation. 1974; 17:331-340.
2. Dexter T M, Wright E G, Krizsa F, Lajtha L G. Regulation of haemopoietic stem cell proliferation in long term bone marrow cultures. Biomedicine. 1977; 27:344-349.
3. Zipori D. Cell interactions in the bone marrow microenvironment: role of endogenous colony-stimulating activity. J Supramol Struct Cell Biochem. 1981; 17:347-357.
4. Noort W A, Kruisselbrink A B, in't Anker P S, et al. Mesenchymal stem cells promote engraftment of human umbilical cord blood-derived CD34(+) cells in NOD/SCID mice. Exp Hematol. 2002; 30:870-878.
5. Prockop D J. Marrow stromal cells as stem cells for nonhematopoietic tissues. Science. 1997; 276:71-74.
6. Hung S C, Pochampally R R, Hsu S C, et al. Short-Term Exposure of Multipotent Stromal Cells to Low Oxygen Increases Their Expression of CX3CR1 and CXCR4 and Their Engraftment In Vivo. PLoS ONE. 2007; 2:e416.
7. Caplan A I, Bruder S P. Mesenchymal stem cells: building blocks for molecular medicine in the 21st century. Trends Mol. Med. 2001; 7:259-264.
8. Barry F P, Murphy J M. Mesenchymal stem cells: clinical applications and biological characterization. Int J Biochem Cell Biol. 2004; 36:568-584.
9. Javazon E H, Beggs K J, Flake A W. Mesenchymal stem cells: paradoxes of passaging. Exp Hematol. 2004; 32:414-425.
10. Bruder S P, Fink D J, Caplan A I. Mesenchymal stem cells in bone development, bone repair, and skeletal regeneration therapy. J Cell Biochem. 1994; 56:283-294.
11. Pittenger M F, Mackay A M, Beck S C, et al. Multilineage potential of adult human mesenchymal stem cells. Science. 1999; 284:143-147.
12. Zipori D, Friedman A, Tamir M, Silverberg D, Malik Z. Cultured mouse marrow cell lines: interactions between fibroblastoid cells and monocytes. J Cell Physiol. 1984; 118:143-152.
13. Zipori D, Duksin D, Tamir M, Argaman A, Toledo J, Malik Z. Cultured mouse marrow stromal cell lines. II. Distinct subtypes differing in morphology, collagen types, myelopoietic factors, and leukemic cell growth modulating activities. J Cell Physiol. 1985; 122:81-90.
14. Benayahu D, Kletter Y, Zipori D, Wientroub S. Bone marrow-derived stromal cell line expressing osteoblastic phenotype in vitro and osteogenic capacity in vivo. J Cell Physiol. 1989; 140:1-7.
15. Jiang Y, Jahagirdar B N, Reinhardt R L, et al. Pluripotency of mesenchymal stem cells derived from adult marrow. Nature. 2002; 418:41-49.
16. Serafini M, Dylla S J, Oki M, et al. Hematopoietic reconstitution by multipotent adult progenitor cells: precursors to long-term hematopoietic stem cells. J Exp Med. 2007; 204:129-139.
17. Kogler G, Sensken S, Airey J A, et al. A new human somatic stem cell from placental cord blood with intrinsic pluripotent differentiation potential. J Exp Med. 2004; 200:123-135.
18. D′Ippolito G, Diabira S, Howard G A, Menei P, Roos B A, Schiller P C. Marrow-isolated adult multilineage inducible (MIAMI) cells, a unique population of postnatal young and old human cells with extensive expansion and differentiation potential. J Cell Sci. 2004; 117:2971-2981. Epub 2004 June 2971.
19. De Coppi P, Bartsch G, Jr., Siddiqui M M, et al. Isolation of amniotic stem cell lines with potential for therapy. Nat. Biotechnol. 2007; 25:100-106.
20. Toma J G, Akhavan M, Fernandes K J, et al. Isolation of multipotent adult stem cells from the dermis of mammalian skin. Nat Cell Biol. 2001; 3:778-784.
21. Anjos-Afonso F, Bonnet D. Nonhematopoietic/endothelial SSEA-1+ cells define the most primitive progenitors in the adult murine bone marrow mesenchymal compartment. Blood. 2007; 109:1298-1306.
22. Muraglia A, Cancedda R, Quarto R. Clonal mesenchymal progenitors from human bone marrow differentiate in vitro according to a hierarchical model. J Cell Sci. 2000; 113 (Pt 7):1161-1166.
23. Zipori D. The stem state: plasticity is essential, whereas self-renewal and hierarchy are optional. Stem Cells. 2005; 23:719-726.
24. Zhang J, Niu C, Ye L, et al. Identification of the haematopoietic stem cell niche and control of the niche size. Nature. 2003; 425:836-841.
25. Scadden D T. The stem-cell niche as an entity of action. Nature. 2006; 441:1075-1079.
26. Taichman R S. Blood and bone: two tissues whose fates are intertwined to create the hematopoietic stem-cell niche. Blood. 2005; 105:2631-2639.
27. Sackstein R, Merzaban J S, Cain D W, Dagia N M, Spencer J A, Lin C P, Wohlgemuth R. 2008. Ex vivo glycan engineering of CD44 programs human multipotent mesenchymal stromal cell trafficking to bone. Nat. Med. 14(2):181-7.
28. Morad V, Pevsner-Fischer M, Barnees S, Samokovlisky A, Rousso-Noori L, Rosenfeld R, Zipori D. 2008. The myelopoietic supportive capacity of mesenchymal stromal cells is uncoupled from multipotency and is influenced by lineage determination and interference with glycosylation. Stem Cells 26(9):2275-86

1. (canceled)

2. A method for increasing mesenychmal stem cell-supported engraftment of hematopoietic stem cells in a subject, the method comprising:
contacting at least a portion of the mesenchymal stem cells with at least one saccharide binding agent;
determining binding of said saccharide binding agent to said mesenchymal stem cells;
determining said glycosylation pattern according to binding of said at least one saccharide binding agent;
correlating said glycosylation pattern to the state of the mesenchymal stem cells; and
selecting mesenchymal stem cells having a glycosylation pattern favorable for supporting engraftment of the hematopoietic stem cells.

3. The method of claim 2, further comprising introducing said selected mesenchymal stem cells directly into the bone marrow of the subject.

4. A method for enhancing hemopoiesis in vitro by mesenchymal stem cells, the method comprising:
contacting at least a portion of the mesenchymal stem cells with at least one saccharide binding agent;
determining binding of said saccharide binding agent to said mesenchymal cells;
determining said glycosylation pattern according to binding of said at least one saccharide binding agent;
correlating said glycosylation pattern to the state of the mesenchymal cells; and selecting mesenchymal stem cells having a glycosylation pattern favorable for supporting hemopoieis.

5. (canceled)

6. The method claim 2, wherein said at least one saccharide-binding agent comprises at least five saccharide-binding agents.

7. The method of claim 2, wherein said contacting of stem cell comprises:
providing on a surface of a substrate a plurality of different essentially sequence- and/or site-specific saccharide-binding agents, which bind saccharide-recognition sequences of a polysaccharide, wherein a number of the plurality of said different essentially sequence- and/or site-specific saccharide binding agents are immobilized on the same surface of said substrate;
contacting said surface with a polysaccharide to be analyzed, or with a mixture comprising a plurality of fragments of said polysaccharide, of the stem cell;
washing or otherwise removing unbound polysaccharide or polysaccharide fragments;
adding to the obtained surface an essentially sequence- and/or site-specific saccharide-binding marker, or a mixture of essentially sequence- and/or site-specific saccharide-binding markers, wherein said marker or mixture of markers binds said bound polysaccharide; and
detecting binding of said saccharide-binding markers that are bound to said surface.

8. The method claim 7, wherein said essentially sequence- and/or site-specific binding agent comprises a lectin.

9. The method of claim 8, wherein said lectin is selected from the group consisting of a colored lectin, a fluorescent lectin, and a biotin labeled lectin.

10. The method of claim 9, wherein said lectin is selected from the group consisting of a complex lectin, a N-acetylglucosamine-binding lectin, a glucosamine/mannose-binding lectin, a mannose-binding lectin, a terminal N-acetylglucosamine-binding lectin, an α-galactose-binding lectin, a terminal β-galactose-binding lectin, a terminal galactose/N-acetyl-galactoseamine-binding lectin, a fucose-binding lectin, and a sialic acid lectin.

11. The method of claim 8, wherein said essentially sequence- and/or site-specific binding agents comprises an antibody.

12. The method of claim 11, wherein said antibody is selected from the group consisting of a fluorescent antibody, a biotin-labeled antibody, and an enzyme-labeled antibody.

13. The method of claim 7, wherein said surface is selected from the group consisting of a bead and an array.

14. The method of claim 7, wherein said detecting binding of said saccharide-binding markers comprises acquiring images of said markers.

15. The method of claim 14, further comprising generating a map of recognition sites of said polysaccharide.

16. The method of claim 7, wherein said marker is selected from the group consisting of a chromogenic binding agent and a labeled binding agent.

17. The method of claim 2, further comprising:
Controlling the differentiation state of the stem cell according to said correlated glycosylation pattern.

18. The method of claim 2, further comprising:
determining when stem cells are suitable for transplantation at least partially according to said correlated glycosylation pattern.

19. The method of claim 2, further comprising:
Determining whether myelopoiesis is supported according to said glycosylation pattern.

20. The method of claim 2, further comprising:
If differentiation has occurred, determining a type of cell into which the stem cell has differentiated at least partially according to said glycosylation pattern.

21-22. (canceled)

23. A method for controlling differentiation of a plurality of stem cells, comprising:
Obtaining a sample from the plurality of stem cells;
Measuring a glycosylation pattern of said sample; and
Treating the plurality of stem cells to control differentiation according to said glycosylation pattern.

24. The method of claim 23, wherein said treating the plurality of stem cells comprises directly inducing a differentiated cell state in the plurality of stem cells.

25. The method of claim 24, wherein said stem cell is selected from the group consisting of a whole stem cell and a non-whole stem cell material.

26. The method of claim 25, wherein said non-whole stem cell material is selected from the group consisting of a membrane protein extract, a homogenized cell, a crude membrane mixture, a crude cell mixture, a non-whole material derived from adding detergent and/or performing solubilization and/or extraction to cells, or mixtures thereof.

27. The method of claim 26 wherein the method is performed in vitro.

28. The method of claim 25, wherein said whole stem cell is fixed.
